(19) United States
(12) Patent Application Publication
(10) Pub. No.: US 2021/0269572 A1
(43) Pub. Date: Sep. 2, 2021

(54) IMPROVING THE MECHANICAL INTEGRITY OF POLYSULFONIC ACIDS

(71) Applicant: UNIVERSITY OF FLORIDA RESEARCH FOUNDATION, INCORPORATED, GAINESVILLE, FL (US)

(72) Inventors: Kenneth B. WAGENER, Gainesville, FL (US); Taylor W. GAINES, Chandler, AZ (US); Michael BELL, North East, MD (US); Julia Grace PRIBYL, Gainesville, FL (US)

(21) Appl. No.: 17/256,356

(22) PCT Filed: Jun. 25, 2019
(86) PCT No.: PCT/US2019/038987

§ 371 Date: Dec. 28, 2020

Related U.S. Application Data
(60) Provisional application No. 62/689,341, filed on Jun. 25, 2018.

(51) Int. Cl. C08F 228/02; C07C 309/67; C08G 61/12; C08F 128/02

(52) U.S. Cl. CPC C08F 228/02; C07C 309/67; C08G 61/12; C08F 128/02; C08G 2261/80; C08G 2261/122; C08G 2261/592; C08G 2261/516; C08G 2261/419; C08G 2261/1452; C08G 2261/135; C08G 2261/724

(57) ABSTRACT

Poly(sulfonic acid)s including a multiplicity of sulfonic acid units separated by alkylene units in a polymer chain or a copolymer chain, the poly(sulfonic acid) having a degree of crosslinking in a range of from about 0.1 to about 30 percent. Methods of preparing poly(sulfonic acid)s having improved mechanical integrity. The methods may include synthesizing a poly(sulfonic acid) by acyclic diene metathesis (ADMET) polymerization and reacting a plurality of double bonds afforded by the ADMET polymerization with a crosslinker. The crosslinking reaction may achieve a degree of crosslinking in a range of from about 0.1 to about 30 percent.

### CROSS-REFERENCE TO RELATED APPLICATION

This application claims the benefit of U.S. Provisional Patent Application No. 62/689,341, filed Jun. 25, 2018, titled “Improving the Mechanical Integrity of Polysulfonic Acids,” which is incorporated by reference herein in its entirety.

### STATEMENT REGARDING FEDERALLY SPONSORED RESEARCH OR DEVELOPMENT

This invention was made with government support under W911 NF-13-1-0362 awarded by ARMY/ARO and DMR1505778 awarded by the National Science Foundation. The government has certain rights in the invention.

### BACKGROUND

Polymers containing precisely-spaced carboxylic, phosphonic, and boronic acids have been synthesized by ADMET; however, until now, precision polymers containing sulfonic acid and sodium sulfonates remained elusive. Each precision acid system has required a different synthetic pathway, a challenge arising from the differences in properties of each acid group. Acidic groups require protection for successful ADMET polymerization. On the other hand, deprotection has been the major challenge in maintaining true precision in polymer structure. A need, therefore, exists for methods of obtaining precision poly(sulfonic acids), as well as for crosslinked precision poly(sulfonic acids) having improved mechanical properties.

### BRIEF SUMMARY

Various embodiments relate to a poly(sulfonic acid) including a multiplicity of sulfonic acid units separated by alkylene units in a polymer chain or a copolymer chain, the poly(sulfonic acid) having a degree of crosslinking in a range of from about 0.1 to about 30 percent.

Various embodiments relate to a method of preparing a poly(sulfonic acid) having improved mechanical integrity. Generally, the method may include synthesizing a poly(sulfonic acid) by acyclic diene metathesis (ADMET) polymerization and reacting a plurality of double bonds afforded by the ADMET polymerization with a crosslinker. The crosslinking reaction may achieve a degree of crosslinking in a range of from about 0.1 to about 30 percent.

### BRIEF DESCRIPTION OF THE FIGURES

Many aspects of this disclosure can be better understood with reference to the following figures, in which:

FIG. 1A is an example according to various embodiments illustrating an ¹H NMR of monomer 4, ethyl tricosa-1,22-diene-12-sulfonate in CDCl₃;

FIG. 1B is an example according to various embodiments illustrating an ¹H NMR of unsaturated ethyl protected polymer SO₃Et21U-33K in CDCl₃;

FIG. 1C is an example according to various embodiments illustrating an ¹H NMR of completely saturated ethyl protected polymer SO₃Et21-33K in CDCl₃;

FIG. 2A is an example according to various embodiments illustrating sulfonic polymer IR spectra for the SO₃Et21U-33K stage of the synthesis;

FIG. 2B is an example according to various embodiments illustrating sulfonic polymer IR spectra for the SO₃Et21-33K stage of the synthesis;

FIG. 2C is an example according to various embodiments illustrating sulfonic polymer IR spectra for the SO₃Na21-33K stage of the synthesis;

FIG. 2D is an example according to various embodiments illustrating sulfonic polymer IR spectra for the SO₃H21-33K stage of the synthesis;

FIG. 3 is an example according to various embodiments illustrating DSC thermogram overlay of SO₃Et21U-33K, SO₃Et21-33K, SO₃Na21-33K, and SO₃H21-33K representing each step of polymer transformation;

FIG. 4A is an example according to various embodiments illustrating DSC thermogram overlay of sodium sulfonate polymers;

FIG. 4B is an example according to various embodiments illustrating DSC thermogram overlay of sulfonic acid polymers. Samples were heated/cooled at 10° C./min;

FIG. 5A is an example according to various embodiments illustrating TGA thermogram overlay of sodium sulfonate polymers; and

FIG. 5B is an example according to various embodiments illustrating TGA thermogram overlay of sulfonic acid polymers.

It should be understood that the various embodiments are not limited to the examples illustrated in the figures.

### DETAILED DESCRIPTION

### Introduction and Definitions

Various embodiments may be understood more readily by reference to the following detailed description. Unless defined otherwise, all technical and scientific terms used herein have the same meaning as commonly understood by one of ordinary skill in the art to which this disclosure belongs.

As used herein, the term “standard temperature and pressure” generally refers to 25° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

The term “mol percent” or “mole percent” generally refers to the percentage that the moles of a particular component are of the total moles that are in a mixture. The sum of the mole fractions for each component in a solution is equal to 1.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

The examples and embodiments described herein are for illustrative purposes only and that various modifications or changes in light thereof will be suggested to persons skilled in the art and are to be included within the spirit and purview of this application. Many variations and modifications may be made to the above-described embodiment(s) of the disclosure without departing substantially from the spirit and principles of the disclosure. All such modifications and variations are intended to be included herein within the scope of this disclosure.

### About Polymers According to Various Embodiments

The present disclosure relates generally to the synthesis of polymers containing precisely spaced sulfonic acid functionalities by acyclic diene metathesis (ADMET) polymerization, and more specifically to providing such polymers with improved mechanic integrity. According to various embodiments, instead of hydrogenating, the double bond, afforded by the ADMET polymerization, may be reacted with a crosslinker to achieve about 0.1 percent to about 30 percent crosslinked materials. In other words, the crosslinked polymers according to any embodiments described herein may have a degree of crosslinking. The degree of crosslinking may be within a range having a lower limit and/or an upper limit. The range may include or exclude the lower limit and/or the upper limit. The lower limit and/or upper limit may be selected from about 0.1, 0.5, 1, 5, 10, 15, 20, 25, 30, 35, 40, 45, and 50%. For example, according to certain embodiments, the degree of crosslinking may be from about 0.1 to about 30%, or any combination of lower limits and upper limits described. This crosslinking may aid in improving the mechanical integrity of the polymer.

### Benefits of Acidic Functionalities

Acidic functionalities enhance polymer properties by increasing strength and toughness, and by allowing for proton conduction. Applications include hydrogels, gas barriers, coatings, and adhesives, to name a few. Fuel cell applications have been of primary interest in recent years due to the expansion of alternative energy research. Commercial sulfonic acid-functionalized materials, such as Nafion®, make excellent proton exchange membranes for fuel cells, because the high acidity of sulfonic groups promotes proton conduction.

Some commercially produced sulfonic acid polymers lack definition and uniformity, inevitable features induced by defects arising from the random nature of most polymerization mechanisms. Also, sulfonation is often performed post-polymerization, and only the surface of the polymer is functionalized or not completely sulfonated. Methods of installing sulfonic acids after the polymer has been made, lack sophistication and control, making precise placement of the sulfonic acid groups difficult or impossible.

According to various embodiments, the precision allowed by ADMET enables precise placement of sulfonate groups, which in turn, may provide more control over morphology, especially the proton conducting domains which exist within the architectures. The sulfonate groups may be converted to sulfonic acid groups. Precision carboxylic acid polymers exist in layers, with the carboxylic groups hydrogen bonded between lamellae. Precision sulfonic acids may behave in the same manner, but with properties which give rise to proton conduction.

### Benefits of Crosslinking

Post-ADMET polymerization, carbon-carbon double bonds can be reacted to provide crosslinking. Reaction of between about 0.1% and about 35% of the double bonds with polymer samples results in a significant improvement in mechanical properties. Crosslinking reactions that can be carried out with the unsaturated polymers include, but are not limited to, free-radical reactions, olefin metathesis with triene molecules, epoxidation followed by addition of various hardeners, thiol-ene and other “click” reactions. Crosslinking can be carried out via: a diacrylate reacting with ADMET double bonds; a dithiol reaction with ADMET double bonds; the epoxidation of the ADMET double bonds followed by diol or diamine addition; bromination of double bonds followed by reaction with a difunctional nucleophilic reagent; or by addition of photoreactive crosslinkers. Alternatively, high energy irradiation of a device prepared from the reduced sulfone, for example, in the form of a membrane, can be carried out to crosslink and to stabilize the membrane. Such a crosslinked membrane, or other device, can be used as a component of a fuel cell or a water desalination device. Combining such a mechanical improvement with the significant thermal properties afforded by the ADMET products will allow these materials to be used in a range of commodity and engineering applications in many forms including fibers and membranes.

### General Properties and Utilities of the Polymers According to Various Embodiments

The polymers, according to various embodiments, in both crosslinked an uncrosslinked states may have utility in a variety of applications.

The polymers may be utilized as membranes for solid-oxide fuel cells, flow batteries, hydrogen pumping, membranes for ion conductivity, membranes for medical use (aliphatic rather than aromatic polymers), and other various applications.

The polymers may be utilized as fibers including hollow fibers, high modulus fibers, and other various applications.

The polymers may be utilized as coatings for various applications. The polymers may be applied as paints or coatings in an uncured and uncrosslinked stated and may be cured or crosslinked once applied.

The polymers may be utilized in a variety of medical applications, including in catheters, stents, and other various applications.

The polymers may be utilized in film wrap, plastic bags, electrical insulation, toys, pipes, siding, flooring, seat covers, packaging, latex paints, adhesives, aircraft applications, automotive applications, additives for blending to alter existing polymers

The polymers may provide superior or improved barrier properties, hardness, tensile strength, creep or time dependent behavior, corrosion resistance, resistance to environmental stress cracking, toughness, strength/modulus to weight ratio, transparency, thermosetting properties, shape memory properties, and others.

The polymers may be useful in “smart” materials that are responsive to the environment to which they are exposed.

### Exemplary Monomer Synthesis Routes

Synthesis of precise sulfonic-acid and sodium sulfonate functionalized polyolefins have benefited from improvements in the monomer synthesis, which have made it possible to produce larger quantities of ester-protected sulfonic acids positioned on every 9^{th} and 21^{st} carbon of the polyolefin backbone to study the effect of acid concentration on morphology. To mimic the product of a conventional polymerization, a random copolymerization was performed using a sulfonate ester and 1,9-decadiene. Following descriptions of an improved protected monomer, novel sulfonate deprotection chemistry is presented, and structural and thermal characteristics of polymers are compared.

Reaction 1 is an example according to various embodiments showing an efficient monomer synthesis route. As shown in Reaction 1, an alkenol with varying methylene spacer lengths (x) may be reacted with trifluoromethanesulfonic anhydride to form a triflate functionalized alkene species (1,2).

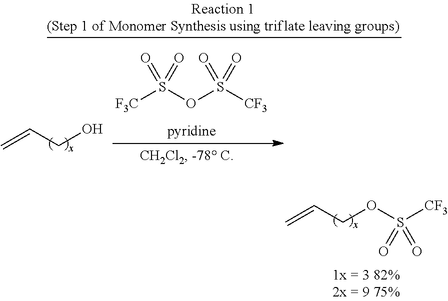

In Reaction 1, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. Reaction 1 may provide a yield of from 5% to 100%.

Reaction 2A is an example according to various embodiments showing a continuation of the efficient monomer synthesis route described according to Reaction 1. As shown in Reaction 2A, LDA may first be added to deprotonate ethyl methane sulfonate, followed by addition of the desired triflate functionalized alkene. A substoichiometric amount of LDA and triflate reagents may be used to avoid trialkylated monomers. This process may be repeated to afford the ethyl protected sulfonate ester diene monomers (3,4).

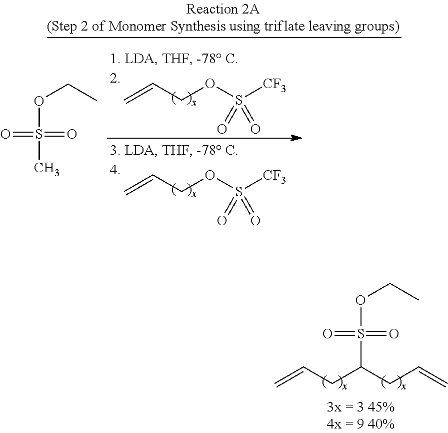

In Reaction 2A, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. Reaction 2A may provide a yield of from 1% to 100%.

Reaction Scheme 2B is an example showing a previously reported monomer synthesis, which resulted in low yields, but which may be used as an alternative to Reaction 2A. In other words, Reaction 2B may be an alternative continuation of the efficient monomer synthesis route described according to Reaction 1. As shown in Reaction 2B, LDA may first be added to deprotonate ethyl methane sulfonate, followed by addition of the desired alkenyl bromide. This process may be repeated to afford the ethyl protected sulfonate ester diene monomers. Low yield may be attributed to the bromide leaving group ability, which may be improved through the use of a triflate leaving group (shown in Reaction 2A).

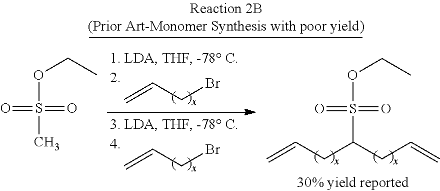

In Reaction 2B, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. Reaction 2B may provide a yield of from 1% to 100%.

### Exemplary Polymerization

Reaction 3 is an example according to various embodiments showing a polymerization reaction scheme utilizing the monomers synthesized according to Reactions 1 and 2A or 2B or as otherwise obtained. As shown in Reaction 3, the sulfonate ester diene monomer may be polymerized via ADMET polymerization using a Grubbs first generation catalyst in dichloromethane at reflux, affording an unsaturated polymer with sulfonate ester groups precisely placed along the polymer backbone.

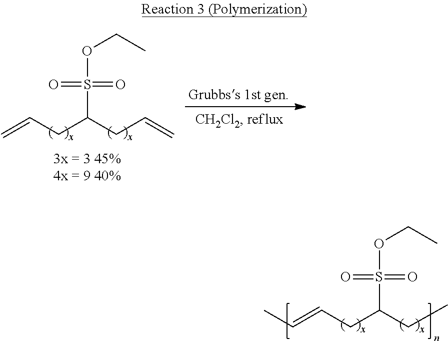

In Reaction 3, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. In Reaction 3, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 3 may provide a yield of from 1% to 100%.

### Exemplary Precision Poly(sulfonic Acid) Synthesis

Reaction 4 is an example according to various embodiments showing a reaction scheme for saturating the double bonds in the polymer product obtained according to Reaction 3. As shown in Reaction 4, the unsaturated polymer with sulfonate ester groups placed with precise spacing along the polymer backbone was dissolved in toluene then hydrogenated using Wilkinson's catalyst in a Parr reactor at 500 psi hydrogen pressure for 5 days.

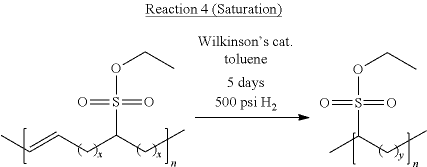

In Reaction 4, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. In Reaction 4, y may be within a range having a lower limit and/or an upper limit. The range may include or exclude the lower limit and/or the upper limit. The lower limit and/or upper limit may be selected from about 2, 5, 10, 15, 20, 25, 30, 35, 40, 45, 50, 55, 60, 65, 70, 75, 80, 85, 90, 95, and 100. For example, according to certain embodiments, y may be from 2 to 100, or any combination of lower limits and upper limits described. In Reaction 4, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 4 may provide a yield of from 1% to 100%.

Reaction 5 is an example according to various embodiments of a deprotection reaction scheme for deprotecting the sulfonate group(s) in the product obtained according to Reaction 4. As shown in Reaction 5, the sulfonate ester may be deprotected in a heterogeneous to homogeneous fashion. The polymer may be mixed with a polar solvent such as ethanol, methanol, water, dimethylsulfoxide, or dimethylformamide along with sodium methoxide, potassium hydroxide, or sodium hydroxide. The mixture may be heated, and as deprotection occurs, the polymer may become more soluble which in turn promotes more deprotection. Once deprotection is complete, the polymer mixture may be a homogeneous solution. As used herein, the term “complete” may, but need not mean that all groups are deprotected; “complete” may mean that a desired or acceptable degree of deprotection is achieved.

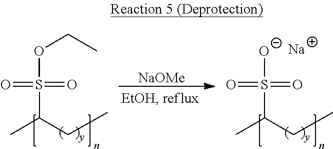

In Reaction 5, y may be within a range having a lower limit and/or an upper limit. The range may include or exclude the lower limit and/or the upper limit. The lower limit and/or upper limit may be selected from about 2, 5, 10, 15, 20, 25, 30, 35, 40, 45, 50, 55, 60, 65, 70, 75, 80, 85, 90, 95, and 100. For example, according to certain embodiments, y may be from 2 to 100, or any combination of lower limits and upper limits described. In Reaction 5, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 5 may provide a yield of from 1% to 100%.

Reaction 6 is an example according to various embodiments of an acidification reaction scheme for acidifying the deprotected sulfonate group(s) in the product obtained according to Reaction 5. As shown in Reaction 6, the deprotected sodium sulfonate polymer may be acidified using concentrated HCl and heated at reflux in ethanol to afford a sulfonic acid groups precisely placed along the polymer backbone.

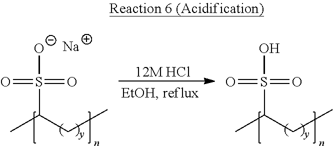

In Reaction 6, y may be within a range having a lower limit and/or an upper limit. The range may include or exclude the lower limit and/or the upper limit. The lower limit and/or upper limit may be selected from about 2, 5, 10, 15, 20, 25, 30, 35, 40, 45, 50, 55, 60, 65, 70, 75, 80, 85, 90, 95, and 100. For example, according to certain embodiments, y may be from 2 to 100, or any combination of lower limits and upper limits described. In Reaction 6, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 6 may provide a yield of from 1% to 100%.

Structure 1 is an example according to various embodiments of a poly(sulfonic acid) that may be obtained, for example as a product of Reaction 6.

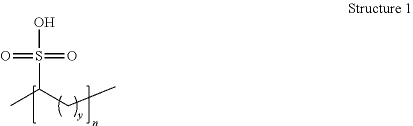

In Structure 1, y may be within a range having a lower limit and/or an upper limit. The range may include or exclude the lower limit and/or the upper limit. The lower limit and/or upper limit may be selected from about 2, 5, 10, 15, 20, 25, 30, 35, 40, 45, 50, 55, 60, 65, 70, 75, 80, 85, 90, 95, and 100. For example, according to certain embodiments, y may be from 2 to 100, or any combination of lower limits and upper limits described. In Structure 1, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described.

### Exemplary Cross-Linked Precision Poly(sulfonic Acid) Synthesis

Reaction 7 is an example according to various embodiments of a deprotection reaction scheme for deprotecting the sulfonate group(s) in the product obtained according to Reaction 3, skipping the saturation step of Reaction 4. As shown in Reaction 7, the sulfonate ester may be deprotected in a heterogeneous to homogeneous fashion. The polymer may be mixed with a polar solvent such as ethanol, methanol, water, dimethylsulfoxide, or dimethylformamide along with sodium methoxide, potassium hydroxide, or sodium hydroxide. The mixture may be heated, and as deprotection occurs, the polymer may become more soluble which in turn promotes more deprotection. Once deprotection is complete, the polymer mixture may be a homogeneous solution. As used herein, the term “complete” may, but need not mean that all groups are deprotected; “complete” may mean that a desired or acceptable degree of deprotection is achieved.

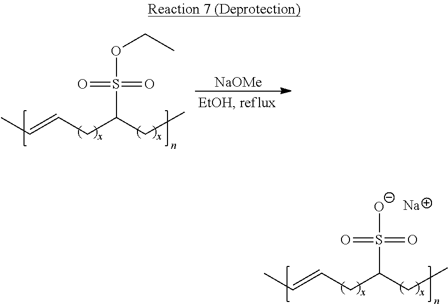

In Reaction 7, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. In Reaction 7, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 7 may provide a yield of from 1% to 100%.

Reaction 8 is an example according to various embodiments of an acidification reaction scheme for acidifying the deprotected sulfonate group(s) in the product obtained according to Reaction 7. As shown in Reaction 8, the deprotected sodium sulfonate polymer may be acidified using concentrated HCl and heated at reflux in ethanol to afford a sulfonic acid groups precisely placed along the polymer backbone.

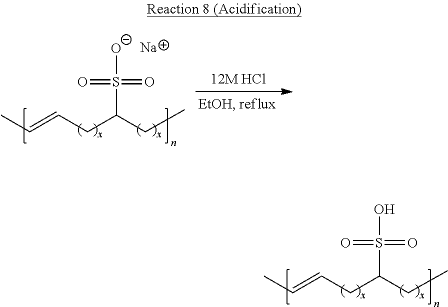

In Reaction 8, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. In Reaction 8, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described. Reaction 8 may provide a yield of from 1% to 100%.

Reaction 9 is an example according to various embodiments of a crosslinking reaction scheme for crosslinking the poly(sulfonic acid) product(s) obtained in from Reaction 8. As shown in Reaction 9, the unsaturated polymer may be crosslinked using a radical initiator which may react with the double bonds in the polymer. The polymer may also be partially hydrogenated, and the remaining double bonds may act as reactive centers which may react with incorporated radical initiators. The crosslinker employed in Reaction 9 or in any similar crosslinking reactions, may be selected from benzoyl peroxide, dicumyl peroxide, azobis(isobutyronitrile), uv-light, heat, or any other of a large variety of cross-linkers, which are readily known to those having ordinary skill in the art. Reaction 8 may provide a yield of from 1% to 100%.

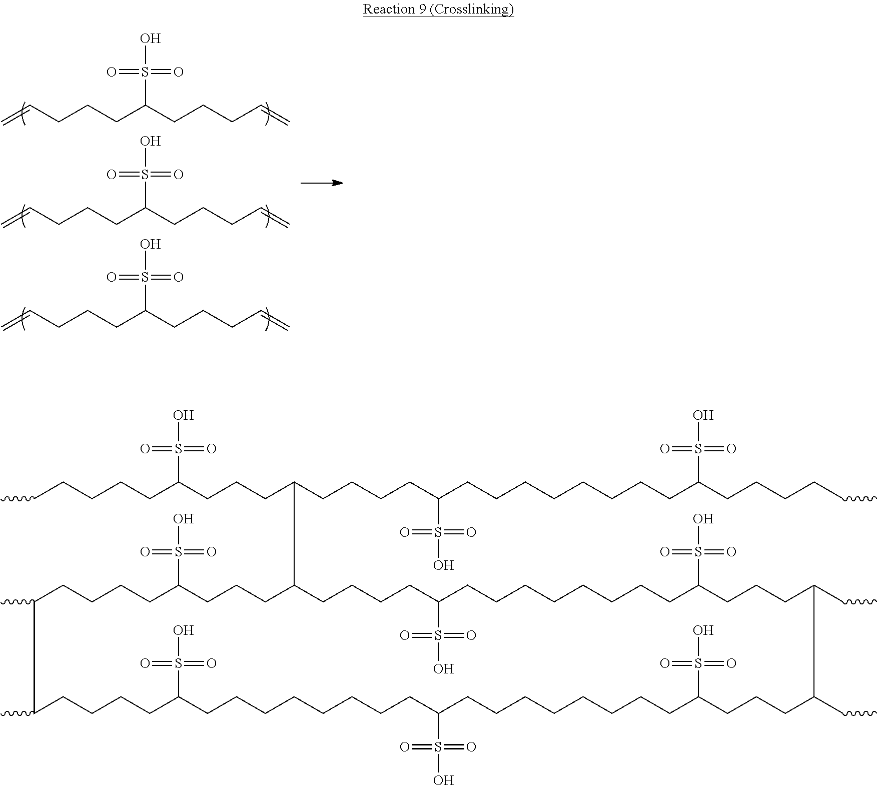

Those having ordinary skill in the art will readily appreciate that although a specific structure is shown as the product of Reaction 9, a variety of structures may be achieved, as illustrated in Structure 2 below.

A generic structure for the crosslinked poly(sulfonic acid)s obtained according to various embodiments, such as via Reaction 9 is shown in Structure 2 below.

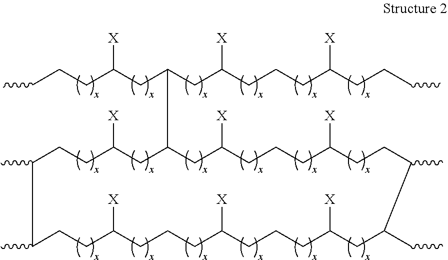

In Structure 2, x may be within a range having a lower limit and/or an upper limit. The range may include or exclude the lower limit and/or the upper limit. The lower limit and/or upper limit may be selected from about 1, 2, 3, 4, 5, 6, 7, 8, 9, 10, 11, 12, 13, 14, 15, 16, 17, 18, 19, 20, 21, 22, 23, 24, and 25. For example, according to certain embodiments, x may be from 1 to 25, or any combination of lower limits and upper limits described. The X functionality may be an acid functionality, such as sulfonic acid, carboxylic acid, phosphonic acid, an others known to those having ordinary skill in the art. The repeat unit shown in Structure 2 may be repeated any number of times. For example, according to various embodiments, the repeat unit of Structure 2 may be repeated n times. According to various embodiments, n may be within a range having a lower limit and/or an upper limit. The range may include or exclude the lower limit and/or the upper limit. The lower limit and/or upper limit may be selected from about 1, 5, 10, 50, 100, 500, 1000, 1500, 2000, 2500, 3000, 3500, 4000, 4500, and 5000. For example, according to certain embodiments, n may be from 1 to 5000, or any combination of lower limits and upper limits described.

### Deprotection Options and Limitations

According to various embodiments, each precision acid system may require a unique synthetic pathway, a challenge arising from the differences in properties of each acid group. Most acidic groups require protection for a successful ADMET polymerization. Various embodiments provide options for achieving maximal deprotection of as many acid groups as possible in order to achieve precision.

Reaction Scheme 10 is an example according to various embodiments showing precise carboxylic acid polymer deprotection. Precisely placed acids were achieved using fairly labile protecting groups. Carboxylic acids were protected with a hemiacetal group.

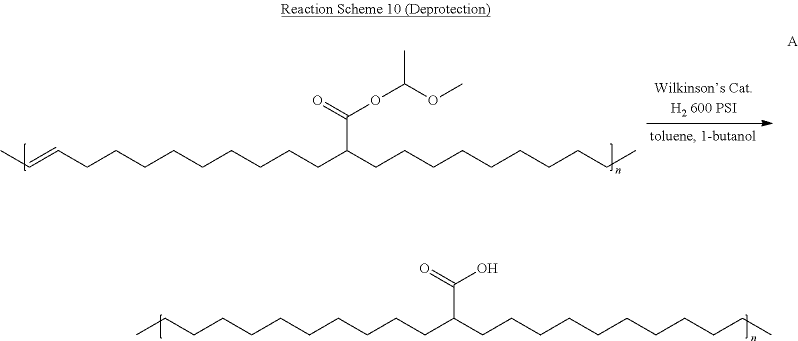

Reaction Scheme 11 is an example according to various embodiments showing precise phosphonic acid deprotection. Phosphonics were protected by an ethyl ester. Boronic acids, a unique case, were synthesized directly utilizing an Ionic liquid and found to be compatible with ADMET polymerization conditions.

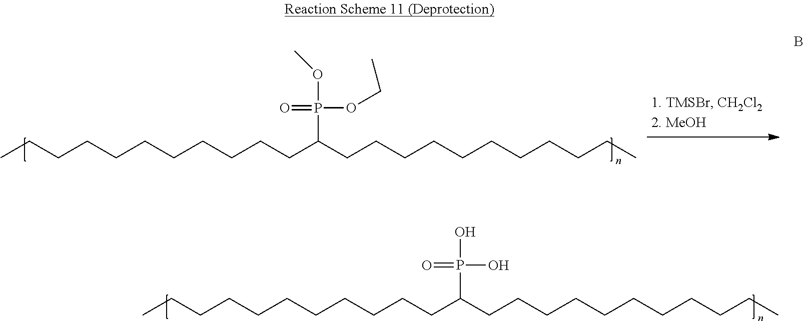

Reaction Scheme 12 is an example according to various embodiments showing prior sulfonic acid deprotection attempts. Reaction Scheme 12 shows protected precision sulfonic acids with a variety of attempted protecting groups. A variety of alternative protecting groups, including neopentyl, isobutyl, and perfluorophenyl have been explored. Alternatives to the directly attached protected ester route were also explored, of which two are notable: precise thiol polymerization followed by post-polymerization oxidation and an aromatic spaced sulfonate ester route. Neither route provided the desired precision sulfonic acids.

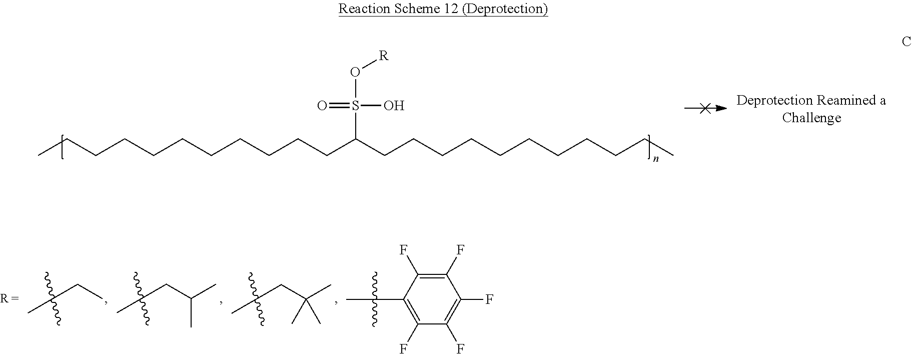

### Some Exemplary Structures

Structures 1, 2, 3, 4, 5, 6, 7, 8, 9, 10, 11, and 12 are examples according to various embodiments illustrating repeat units for precise and random polymer structures. As used herein, the term “sulfonate” refers to a salt or ester of a sulfonic acid. The molecular weight of any of the polymers illustrated in Structures 1, 2, 3, 4, 5, 6, 7, 8, 9, 10, 11, or 12 may be within a range having a lower limit and/or an upper limit. The range may include or exclude the lower limit and/or the upper limit. The lower limit and/or upper limit may be selected from about 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 60,000, 70,000, 80,000, 90,000, 100,000, 150,000, 200,000, 250,000, 300,000, 350,000, 400,000, 450,000, and 500,000 Daltons. For example, according to certain embodiments, the molecular weight of any of the polymers illustrated in Structures 1, 2, 3, 4, 5, 6, 7, 8, 9, 10, 11, or 12 may be about 6,000 Daltons, about 19,000 Daltons, about 33,000 Daltons, from about 6,000 to about 33,000 Daltons, or any combination of lower limits and upper limits described.

Structure 3 is an example according to various embodiments illustrating a repeat unit of polymer structure having a precisely-placed, protected sulfonate group. SO₃Et denotes ethyl sulfonate. The character “U” indicates that the polymer is an ADMET product containing unsaturated double bonds. The number “9” indicates that, in the overall chain, each protected sulfonate group is separated from other protected sulfonate groups by 9 carbons. In other words, 9 indicates a protected sulfonate group may be present on every 9th carbon in the overall structure. The protected sulfonate group may be placed on any carbon in Structure 3. Any number of protected sulfonate groups may be present based on the number of carbon atoms in the repeat unit. The double-bond may be placed between any two carbons in Structure 3. Any number of double-bonds may be present in the repeat unit based on the number of carbon atoms in the repeat unit. The character “n” indicates that the bracketed segment may repeat n times. According to various embodiments, n may be in a range of from XX to XX.

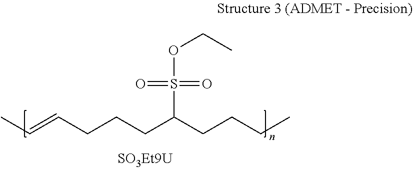

Structure 4 is an example according to various embodiments illustrating a repeat unit of polymer structure having a precisely-placed, protected sulfonate group. SO₃Et denotes ethyl sulfonate. The character “U” indicates that the polymer is an ADMET product containing unsaturated double bonds. The number “21” indicates that, in the overall chain, each protected sulfonate group is separated from other protected sulfonate groups by 21 carbons. In other words, the number “21” indicates a protected sulfonate group may be present on every 21th carbon in the overall structure. The protected sulfonate group may be placed on any carbon in Structure 4. Any number of protected sulfonate groups may be present based on the number of carbon atoms in the repeat unit. The double-bond may be placed between any two carbons in Structure 4. Any number of double-bonds may be present in the repeat unit based on the number of carbon atoms in the repeat unit. The character “n” indicates that the bracketed segment may repeat n times. According to various embodiments, n may be in a range of from XX to XX. The dash and number at the end indicates an exemplary molecular weight, as described above.

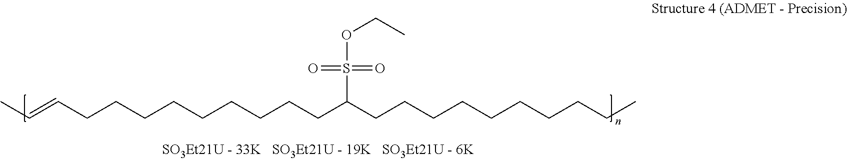

Structure 5 is an example according to various embodiments illustrating a repeat unit of a copolymer structure having a randomly-placed, protected sulfonate group. SO₃Et denotes ethyl sulfonate. “Co” denotes a copolymer made to mimic the sulfonate concentration of Structure 5, but with random sulfonate placement. The character “n” indicates that the first bracketed segment may repeat n times. The sulfonate group may be placed on any carbon within the first bracketed section. Any number of protected sulfonate groups may be present based on the number of carbon atoms in the repeat unit. A first double-bond may be placed between any two carbons within the first bracketed section. Any number of double-bonds may be present in the first bracketed section based on the number of carbon atoms in the first bracketed section. According to various embodiments, n may be in a range of from XX to XX. The character “m” indicates that the second bracketed segment may repeat m times. A second double-bond may be placed between any two carbons within the second bracketed section. Any number of double-bonds may be present in the second bracketed section based on the number of carbon atoms in the second bracketed section. According to various embodiments, m may be in a range of from XX to XX.

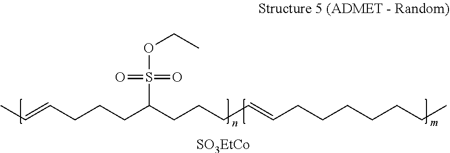

Structure 6 is an example according to various embodiments illustrating the repeat unit of the polymer structure as illustrated in Structure 3 after saturation of the double-bond. The double-bond(s) may be saturated by any method, including any method described herein.

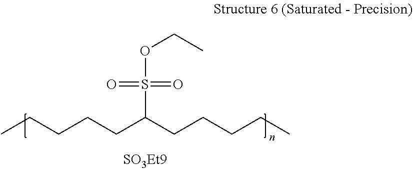

Structure 7 is an example according to various embodiments illustrating the repeat unit of the polymer structure as illustrated in Structure 4 after saturation of the double-bond. The double-bond(s) may be saturated by any method, including any method described herein.

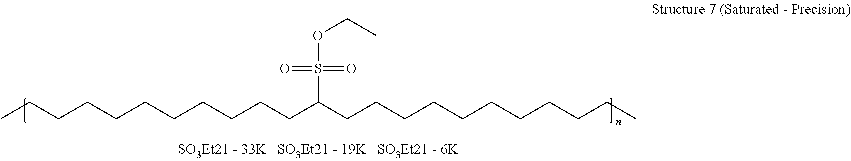

Structure 8 is an example according to various embodiments illustrating the repeat unit of the polymer structure as illustrated in Structure 5 after saturation of the double-bond. The double-bond(s) may be saturated by any method, including any method described herein.

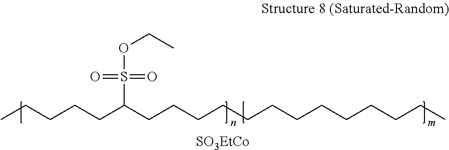

Structure 9 is an example according to various embodiments illustrating the repeat unit of the saturated polymer structure as illustrated in Structure 6 after deprotection of the sulfonate group. The sulfonate group(s) may be deprotected by any method, including any method described herein. SO₃Na denotes sodium sulfonate.

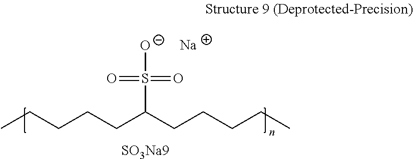

Structure 10 is an example according to various embodiments illustrating the repeat unit of the saturated polymer structure as illustrated in Structure 7 after deprotection of the sulfonate group. The sulfonate group(s) may be deprotected by any method, including any method described herein. SO₃Na denotes sodium sulfonate.

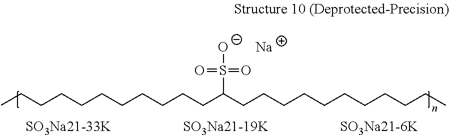

Structure 11 is an example according to various embodiments illustrating the repeat unit of the saturated polymer structure as illustrated in Structure 8 after deprotection of the sulfonate group. The sulfonate group(s) may be deprotected by any method, including any method described herein. SO₃Na denotes sodium sulfonate.

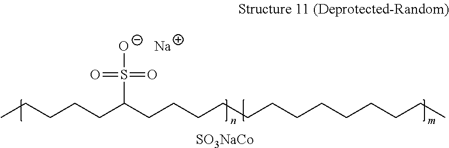

Structure 12 is an example according to various embodiments illustrating the repeat unit of a saturated and deprotected polymer structure as illustrated in Structure 9 after acidification of the deprotected sulfonate group to yield a sulfonic acid polymer. The deprotected sulfonate group(s) may acidified by any method, including any method described herein. SO₃H denotes sulfonic acid.

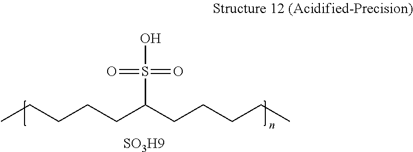

Structure 13 is an example according to various embodiments illustrating the repeat unit of a saturated and deprotected polymer structure as illustrated in Structure 10 after acidification of the sulfonate group to yield a sulfonic acid polymer. The deprotected sulfonate group(s) may acidified by any method, including any method described herein. SO₃H denotes sulfonic acid.

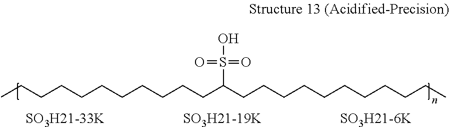

Structure 14 is an example according to various embodiments illustrating the repeat unit of a saturated and deprotected precise sulfonic acid polymer structure as illustrated in Structure 11 after acidification of the sulfonate group to yield a sulfonic acid polymer. The deprotected sulfonate group(s) may acidified by any method, including any method described herein. SO₃H denotes sulfonic acid.

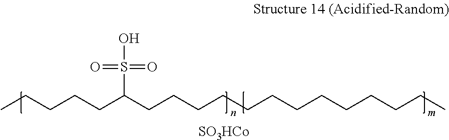

### EXAMPLES

### Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for.

The purpose of the following examples is not to limit the scope of the various embodiments, but merely to provide examples illustrating specific embodiments.

### Methods

All chemicals and materials were purchased from Sigma Aldrich and used as received, unless otherwise stated. Dry solvents were obtained from a solvent purification system. Grubbs' 1st generation catalyst was graciously donated by Materia, Inc. and used as received. Flash chromatography was performed using SiliCycle SiliaFlash® P60, 40-63 μm, 60 Å silica. ¹H NMR and ¹³C NMR spectra were acquired on a Varian Mercury-300 NMR Spectrometer using VNMRJ software. IR spectra were obtained on a PerkinElmer FT-IR Spectrum One with ATR attachment using Spectrum Software for data analysis. Mass spectroscopy was performed in the Department of Chemistry's Mass Spectroscopy Laboratories at the University of Florida. Elemental Analysis was performed by Atlantic Microlabs. Molecular weights were obtained in THF at 40° C. relative to polystyrene standards using an Agilent 1100 GPC with a refractive index detector. Thermogravimetric Analysis (TGA) was performed on a TA Instruments Q5000 using a temperature ramp of 10° C./min under nitrogen atmosphere. Differential Scanning calorimetry (DSC) was performed on a TA Instruments Q1000 DSC. Hermetically sealed aluminum pans were equilibrated at −80° C. and subsequently heated at 10° C./min until the desired final temperature was reached. Pans were then cooled at 10° C./min to −80° C. Three of these heat/cool cycles were performed for each sample. Data are reported for the third cycles, which were reproducible.

### Premonomer Synthesis

### Pent-4-en-1-yl Trifluoromethanesulfonate (1)

To a dry 1 L round bottom flask containing 500 mL of dry DCM, 16 mL of dry pyridine was added. Then 47.68 grams (169 mmols) of trifluoromethanesulfonic anhydride was added dropwise over 15 mins at room temperature. The resulting reagent was stirred and cooled to 0° C. before 12.66 grams (147 mmols) of 4-penten-1-ol was added dropwise. The reaction was allowed to warm to room temperature and was stirred for 1 hour, at which point pyridinium triflate had precipitated. The precipitate was filtered, washed with dry DCM and discarded. The filtrate was concentrated and passed through a plug of silica (9:1, hexanes:DCM). The product flask was quickly purged with argon and placed into a freezer, while NMR spectra were acquired for confirmation. The triflate was subsequently used immediately to prevent degradation. Yield: 23.3 grams, 82%. ¹H NMR (300 MHz, CDCl₃) δ 5.84-5.70 (m, 1H), 5.13-5.05 (m, 2H), 4.55 (t, 2H), 2.20 (q, 2H), 1.94 (p, 2H). ¹³C NMR (75 MHz, CDCl₃) δ 135.7, 120.7, 116.6, 76.7, 63.8, 29.0, 28.3.

### Undec-10-en-1-yl Trifluoromethanesulfonate (2)

The procedure used for pent-4-en-1-yl trifluoromethanesulfonate above was employed: 16 mL of pyridine, 47.68 grams (169 mmols) of trifluoromethanesulfonic anhydride, and 25.03 (147 mmols) grams of 10-undecen-1-ol. Yield: 33.8 grams, 76%. ¹H NMR (300 MHz, CDCl₃) δ 5.86-5.76 (m, 1H), 5.03-4.91 (m, 2H), 4.53 (t, 2H), 2.03 (q, 2H), 1.83 (m, 2H), 1.45-1.27 (br, 12H). ¹³C NMR (75 MHz, CDCl₃) δ 139.1, 120.8, 116.5, 114.1, 76.5, 33.7, 31.6, 29.3, 29.2, 29.0, 28.9, 28.8, 25.0.

### Monomer Synthesis

To a three-neck round bottom flask, 5.00 grams (40.27 mmols) of ethyl methanesulfonate was added and dissolved in 40 mL of dry THF. The solution was cooled to −78° C., approximately 39 mmols of freshly prepared LDA was added dropwise, and the solution was stirred for 15 minutes. The temperature was then raised to 0° C. for 30 minutes to allow for thorough deprotonation. The flask was then lowered again into a −78° C. bath and stirred for 15 minutes before 39 mmols of the appropriate triflate was added dropwise in 50 mL of dry heptane. The reaction was raised to 0° C. for 30 minutes and then lowered back to −78° C. before repeating the addition of LDA and triflate once more to yield dialkylated product. Afterwards, the reaction was concentrated to half the original volume, flooded with deionized water, and extracted with diethyl ether (4×25 mL). The organic layer was collected and dried over MgSO₄. The MgSO₄ was filtered, washed with ether, and discarded, while the filtrate was collected and concentrated to yield crude oil-like products. Products were purified via column chromatography with an eluent mixture consisting of hexanes and ethyl acetate (19:1).

### Ethyl undeca-1,10-diene-6-sulfonate (3)

8.51 grams of pent-4-en-1-yl trifluoromethanesulfonate (1) was added after each deprotonation. Yield: 4.57 grams, 45%. ¹H NMR (300 MHz, CDCl₃) δ 5.83-5.74 (m, 2H), 5.07-4.96 (m, 4H), 4.32-4.25 (q, 2H), 2.99-2.97 (p, 1H), 2.13-2.05 (q, 4H), 1.98-1.88 (m, 4H), 1.75-1.53 (m, 4H), 1.42-1.37 (t, 3H). ¹³C NMR (75 MHz, CDCl₃) δ 137.7, 115.3, 65.4, 60.9, 33.4, 28.3, 25.7, 15.2. HRMS (ESI) (m/z): (M+Na)±calcd for C₂₅H₄₈O₃S 451.3216; found 451.3213. Elemental Analysis: calcd for C₂₅H₄₈O₃S, C: 70.04, H: 11.29, N: 0.00; found C: 69.84, H: 11.55, N: 0.00.

### Ethyl tricosa-1,22-diene-12-sulfonate (4)

11.78 grams of undec-10-en-1-yl trifluoromethanesulfonate (2) was added after each deprotonation. Yield: 6.79 grams, 40%. ¹H NMR (300 MHz, CDCl₃) δ 5.85-5.74 (m, 2H), 5.01-4.91 (m, 4H), 4.31-4.24 (q, 2H), 2.98-2.94 (p, 1H), 2.07-1.97 (q, 4H), 1.95-1.85 (m, 4H), 1.74-1.62 (m, 4H), 1.54-1.28 (br, 27H). ¹³C NMR (75 MHz, CDCl₃) δ 139.2, 114.1, 71.9, 65.3, 61.2, 33.8, 29.5, 29.4, 29.3, 29.1, 28.9, 28.9, 26.6, 15.2. HRMS (ESI) (m/z): (M+NH₄)±calcd for C₁₃H₂₄O₃S 278.1784; found 278.1786. Elemental Analysis: calcd for C₁₃H₂₄O₃S, C: 59.96, H: 9.29, N: 0.00; found C: 60.23, H: 9.42, N: 0.00.

### Polymerization Procedures

Monomer solutions in DCM (2.0M) were inserted into dry Schlenk tubes and subjected to freeze-pump-thaw cycles until gas evolution failed to appear during thaw cycles. A final freeze was performed and while under argon purge, 1 mol % of Grubbs' first generation catalyst was added. The Schlenk was then equipped with a reflux condenser and argon flow adapter. The apparatus was lowered into an oil both at the appropriate temperature to maintain the reflux of DCM. Polymerizations were continued for the times specified individually below, after which samples cooled and a solution of ethyl vinyl ether in toluene (1:10) was added. The polymers were then precipitated from methanol at around −18° C. and subsequently filtered, collected, and dried under high vacuum.

### SO3Et8U.

1.0 gram of ethyl undeca-1,10-diene-6-sulfonate (3). Polymerization proceeded for 72 hours. ¹H NMR (300 MHz, CDCl₃) δ 5.45-5.33 (br, 2H), 4.30-4.23 (q, 4H), 3.01-2.93 (p, 1H), 2.08-1.94 (m, 4H), 1.93-1.84 (m, 4H), 1.78-1.45 (m, 4H), 1.43-1.34 (t, 3H). ¹³C NMR (75 MHz, CDCl₃) δ 130.1, 129.6, 65.5, 60.9, 32.3, 28.4, 27.0, 26.4, 15.3. FT-IR (ATR) v in cm⁻¹ 2935, 2866, 1459, 1391, 1341, 1163, 1097, 1002, 970, 909, 763, 701, 627. GPC (THF, Polystyrene Standards): Mₙ=6,600 g/mol; Mₙ=13,500 g/mol (PDI=2.05).

### SO₃Et20U-33K

2.0 grams of ethyl tricosa-1,22-diene-12-sulfonate (4). Polymerization proceeded for 72 hours. ¹H NMR (300 MHz, CDCl₃) δ 5.39-5.34 (br, 2H), 4.31-4.23 (q, 2H), 2.98-2.94 (p, 1H), 2.02-1.85 (m, 4H), 1.73-1.61 (m, 4H), 1.49-1.24 (br, 27H). ¹³C NMR (75 MHz, CDCl₃) δ 130.3, 65.3, 61.2, 32.6, 29.7, 29.5, 29.5, 29.5, 29.3, 29.2, 28.9, 26.6, 15.2. FT-IR (ATR) v in cm⁻¹ 2922, 2852, 1645, 1464, 1342, 1167, 1095, 1005, 967, 912, 768, 721, 628. GPC (THF, Polystyrene Standards): Mₙ=33,300; Mₙ=73,600 (PDI=2.21).

### SO₃Et20U-19K.

2.0 grams of ethyl tricosa-1,22-diene-12-sulfonate (4). Polymerization proceeded for 24 hours. ¹H NMR (300 MHz, CDCl₃) δ 5.39-5.34, 4.28-4.23 (q, 2H), 2.98-2.92 (p, 1H), 2.01-1.85 (m, 4H), 1.73-1.61 (m, 4H), 1.49-1.26 (br, 27H). ¹³C NMR (75 MHz, CDCl₃) δ 130.3, 65.3, 61.2, 32.6, 29.7, 29.5, 29.5, 29.5, 29.3, 29.2, 28.9, 26.6, 15.2. FT-IR (ATR) v in cm⁻¹ 2922, 2852, 1464, 1342, 1167, 1097, 1005, 967, 912, 768, 721. GPC (THF, Polystyrene Standards): Mₙ=19,800; Mₙ=48,500 (PDI=2.45).

### SO₃Et20U-6K.

2.0 grams of ethyl tricosa-1,22-diene-12-sulfonate (4). Polymerization proceeded for 12 hours. ¹H NMR (300 MHz, CDCl₃) δ 5.39-5.34 (br, 2H), 4.28-4.23 (q, 2H), 2.98-2.92 (p, 1H), 2.01-1.85 (m, 4H), 1.73-1.61 (m, 4H), 1.49-1.26 (br, 27H). ¹³C NMR (75 MHz, CDCl₃) δ 130.3, 65.3, 61.2, 32.6, 29.7, 29.5, 29.5, 29.5, 29.3, 29.2, 28.9, 26.6, 15.2. FT-IR (ATR) v in cm⁻¹ 2915, 2849, 1468, 1342, 1262, 1163, 1100, 1004, 914, 796, 773, 719, 701, 627. GPC (THF, Polystyrene Standards): Mₙ=6,900; Mₙ=15,100 (PDI=2.19).

### SO₃EtCoU.

1.664 grams of ethyl undeca-1,10-diene-6-sulfonate (3) and 1.328 grams of 1,9-decadiene were polymerized for 72 hours. ¹H NMR (300 MHz, CDCl₃) δ 5.48-5.5.30 (br, 4H), 4.31-4.24 (q, 2H), 3.02-2.96 (p, 1H), 2.17-1.86 (br, 8H), 1.78-1.61 (br, 4H), 1.59-1.45 (m, 4H), 1.42-1.25 (br, 11H). ¹³C NMR (75 MHz, CDCl₃) δ 131.5, 130.3, 128.9, 65.3, 61.0, 32.6, 32.3, 29.6, 29.0, 28.3, 27.2, 26.4, 15.2. FT-IR (ATR) v in cm⁻¹ 2923, 2852, 1457, 1342, 1166, 1004, 966, 912, 763, 703. GPC (THF, Polystyrene Standards): Mₙ=2,200; Mₙ=3,200 (PDI=1.45).

### Hydrogenation Procedures

Dry unsaturated polymer samples (1.0 g) were dissolved in 30-50 mL of dry toluene in a round bottom flask and degassed with a steady argon flow for a minimum of 24 hours. Next, 0.5 mol % of Wilkinson's Catalyst (tris(triphenylphosphine)rhodium chloride) was added and immediately the flasks were sealed in a Parr bomb rated for 2000 psi of hydrogen gas. The vessel was purged three times with hydrogen. On the final fill, a pressure of 500 psi of hydrogen was added and the vessel was lowered into an oil bath at 90° C. for 5 days. NMR was performed to confirm complete saturation before the polymers were precipitated from methanol at around −18° C. and subsequently filtered, collected, and dried under high vacuum.

### SO₃Et8.

¹H NMR (300 MHz, CDCl₃) δ 4.31-4.23 (q, 2H), 2.99-2.93 (p, 1H), 1.96-1.84 (m, 4H), 1.73-1.61 (m, 4H), 1.46-1.24 (br, 11H). ¹³C NMR (75 MHz, CDCl₃) δ 65.3, 61.1, 29.5, 29.2, 28.9, 26.6, 15.3. FT-IR (ATR) v in cm⁻¹ 2921, 2852, 1638, 1464, 1340, 1261, 1164, 1096, 1003, 910, 767, 701, 628.

### SO₃Et20-33K.

¹H NMR (300 MHz, CDCl₃) δ 4.31-4.24 (q, 2H), 2.96-2.94 (p, 1H), 1.95-1.85 (m, 4H), 1.72-1.62 (m, 4H), 1.48-1.25 (br, 35H). ¹³C NMR (75 MHz, CDCl₃) δ 65.3, 61.1, 29.7, 29.7, 29.6, 29.6, 29.5, 29.3, 28.9, 26.6, 15.2. FT-IR (ATR) v in cm⁻¹ 2916, 2849, 1467, 1342, 1165, 1099, 1003, 913, 772, 720.

### SO₃Et20-19K.

¹H NMR (300 MHz, CDCl₃) δ 4.32-4.25 (q, 2H), 2.96-2.94 (p, 1H), 1.94-1.83 (m, 4H), 1.72-1.62 (m, 4H), 1.48-1.25 (br, 35H). ¹³C NMR (75 MHz, CDCl₃) δ 65.4, 61.0, 29.8, 29.7, 29.6, 29.5, 29.5, 29.3, 28.9, 26.6, 15.2. FT-IR (ATR) v in cm⁻¹ 2915, 2848, 1467, 1340, 1164, 1099, 1003, 912, 772, 710.

### SO₃Et20-6K.

¹H NMR (300 MHz, CDCl₃) δ 4.30-4.25 (q, 2H), 2.97-2.94 (p, 1H), 1.92-1.81 (m, 4H), 1.72-1.62 (m, 4H), 1.48-1.25 (br, 35H). ¹³C NMR (75 MHz, CDCl₃) δ 65.4, 61.0, 29.8, 29.7, 29.6, 29.5, 29.5, 29.3, 28.9, 26.6, 15.2. FT-IR (ATR) v in cm⁻¹ 2915, 2849, 1467, 1343, 1165, 1100, 1003, 914, 772, 719.

### SO₃EtCo.

¹H NMR (300 MHz, CDCl₃) δ 4.28-4.21 (q, 2H), 2.95-2.91 (p, 1H), 1.90-1.82, (m, 4H), 1.68-1.61 (m, 4H), 1.44-1.33 (t, 3H), 1.29-1.06 (br, 24H). ¹³C NMR (75 MHz, CDCl₃) δ 13C NMR (75 MHz, CDCl₃) δ 65.2, 61.1, 34.3, 29.6, 29.4, 29.3, 28.8, 26.5, 15.2. FT-IR (ATR) v in cm⁻¹ 2917, 2849, 1463, 1342, 1262, 1167, 1096, 1005, 912, 768, 729, 720.

### Deprotection

Dry saturated polymer samples (800 mg) were suspended in 10-15 mL of 200 proof ethanol in a round bottom flask. 5 mL of a 25 wt. % sodium methoxide in methanol solution was added to the mixture and the reaction was allowed to reflux for 72 hours under argon. The reactions were then cooled and concentrated. Flasks were then flooded with cold deionized water and the polymers filtered, washed with water twice more with water, collected, and dried under high vacuum to yield the sodium sulfonate salt precision polymers.

### SO₃Na8.

FT-IR (ATR) v in cm⁻¹ 2923, 2853, 1686, 1436, 1169, 1046, 881, 842, 802, 721, 628.

### SO₃Na20-33K.

FT-IR (ATR) v in cm⁻¹ 3424, 2916, 2849, 1688, 1466, 1436, 1167, 1139, 1051, 880, 842, 803, 723, 631.

### SO₃Na20-19K.

FT-IR (ATR) v in cm⁻¹ 3440, 2916, 2849, 1693, 1467, 1168, 1049, 841, 802, 721, 631.

### SO₃Na20-6K.

FT-IR (ATR) v in cm⁻¹ 3443, 2916, 2849, 1693, 1467, 1165, 1047, 719, 631.

### SO₃NaCo.

FT-IR (ATR) v in cm⁻¹ 3433, 2917, 2850, 1688, 1466, 1170, 1047, 843, 802, 720, 631.

### Acidification

Sodium sulfonate polymer samples (400 mg) were suspended in 5-10 mL of 200 proof ethanol and 5 mL of 12 molar hydrochloric acid was added dropwise while stirring. The mixtures were allowed to reflux for 24 hours under argon. After the reactions were cooled and concentrated. The flasks were then flooded with a cold 2 molar hydrochloric acid solution. Polymers were filtered, washed twice more with the acid solution, and the sulfonic acid polymers were collected and dried under vacuum.

### SO₃H8.

FT-IR (ATR) v in cm⁻¹ 3381, 2923, 2853, 2337, 1646, 1466, 1147, 1034, 714, 629, 606.

### SO₃H20-33K.

FT-IR (ATR) v in cm⁻¹ 2916, 2849, 1700, 1467, 1128, 1034, 851, 805, 719, 628.

### SO₃H20-19K.

FT-IR (ATR) v in cm⁻¹ 2917, 2849, 1700, 1467, 1128, 1031, 917, 719.

### SO₃H20-6K.

FT-IR (ATR) v in cm⁻¹ 2916, 2849, 1700, 1467, 1120, 1032, 804, 719.

### SO₃HCo.

FT-IR (ATR) v in cm⁻¹ 2916, 2849, 1668, 1466, 1156, 1035, 912, 718.

### Results and Discussion

Precise synthesis of protected ester monomers is the most intensive step of the entire route due to the stringent reaction conditions and reagent preparations. Previous literature reported a 30% yield of monomer. However, repeated attempts to reproduce this reaction were not successful to the extent of 30%. Thus, alternatives to the published synthesis were investigated. Alternative syntheses were not successful, and the ethyl sulfonate ester appeared to be the simplest monomer in terms of synthetic steps and reagent preparations. Therefore, the ethyl protecting group was the starting point.

An evaluation of the published monomer reaction conditions was first conducted. Via carbon-carbon bond formation using lithium diisopropylamide (LDA), the optimum monomer synthesis is one-step using commercially available reagents. LDA is added to the ethyl methanesulfonate, resulting in deprotonation of the methyl directly attached to sulfur. The resulting carbanion can then perform a nucleophilic attack on the alkyl bromide of choice; repeating this process will give the α,ω-diene monomer.

Confirmation of ethyl methanesulfonate's deprotonation was scrutinized first, as this is the initial mechanistic step of the reaction. Deprotonation indeed was occurring and confirmed by ¹H NMR. After ethyl methane sulfonate was deprotonated with LDA, the reaction was quenched with deuterium oxide. This process suppressed the methyl peak after deuteration, thus confirming a proton exchange took place with LDA. Since deprotonation was occurring, the substitution reaction was being hindered by some other phenomenon.

Reaction Scheme 2B, above, shows previously reported monomer synthesis. Initially, it was speculated that lithium cations from LDA were bound tightly to the deprotonated methane sulfonate species, preventing bromide displacement and resulting in low yields. However, low yields were observed when alternative counterions, such as potassium, and crown ethers, were employed in an attempt to free the carbanion for nucleophilic attack on the alkyl halide.

The only plausible explanation left was leaving group ability. In fact, success was found when the bromide was abandoned for the triflate leaving group. The monomer yield was improved from 1-3% to 40% and 45% simply by opting for a better leaving group. This new, higher yielding synthetic scheme is shown in Reaction Scheme 2A. More specifically, Reaction 2A shows precision sulfonic acid and sodium salt polymer synthetic route. The new protected sulfonic acid monomer route using triflate leaving groups is faster and results in higher yields.

Triflates 1 and 2 were synthesized by the reaction of commercially available terminal alkene-containing alcohols with trifluoromethanesulfonic anhydride and pyridine. Reagent addition order is key: the reaction between trifluoromethanesulfonic anhydride and pyridine must take place before the alcohol is added, or side reactions result in isomerization of the double bond; isomerization will lead to an imprecise material defeating the purpose of ADMET. The triflates were passed through silica plugs and following structural confirmation were kept inert before use in the next step.

Although monomer synthesis conditions were altered, LDA remained the base of choice. Purification and preparation of LDA starting materials is quite simple, and titration of the base is trivial. A substoichiometric amount (39 mmols) of LDA was used to deprotonate 40 mmols of ethyl methane sulfonate, followed by the addition of the appropriate triflate. Typically, excesses of such bases and reagents would be used to account for residual moisture and enhance yields. However, the use of excess of LDA and triflate reagents resulted in undetectable amounts of trialkylated monomers. These triene (triflunctional) species were in one case carried through purification and even passed elemental and NMR analysis. Resulting polymers from these hidden triene species were crosslinked and consequently imprecise. Consequently, substoichiometric amounts of LDA and triflate were used to avoid triene species formation.

Care was also essential when adding triflates to deprotonated ethane methanesulfonate, which is in tetrahydrofuran (THF). The triflate must be added to the reaction in solution due to its reactivity, yet THF is the wrong solvent choice. Even at low temperature, the triflate was found to cationically ring-open THF, as observed by previous researchers. Dry heptane was instead found to be the ideal solvent for the triflate solution addition, but the temperature was held at −78° C. as a precaution due to the reactivity of triflates.

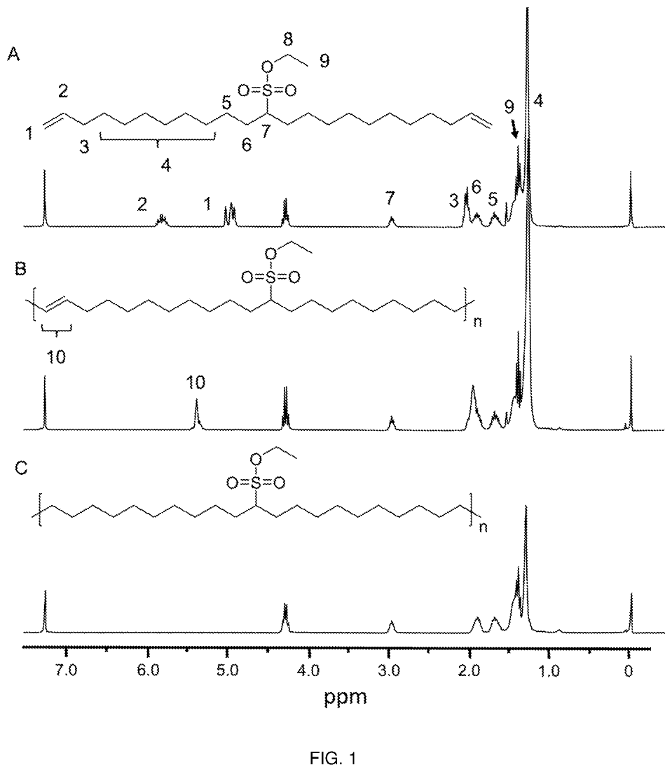

Monomers were purified via column chromatography and characterization was consistent with published results for the ester protected monomer. ¹H NMR of monomer 4 is shown in FIG. 1A. Clearly, the external olefins are intact and isomerization-free at 5.85-5.74 ppm (internal —CH═C—) and 5.01-4.91 (external —C═CH₂). After exposure to ADMET conditions, where the monomers are refluxed in DCM along with Grubbs' first generation catalyst, conversion to polymer is unquestionable. External double bonds are transformed into a single internal olefin signal which resonates at 5.39-5.34 ppm (internal —CH═CH—) with no end-groups detectible via NMR, an indication of high-polymer. Gel permeation chromatography (GPC) results are consistent with this finding. Number-average molecular weights of up to 33,300 g/mol were achievable after 72 hours of polymerization, at which time solutions were highly viscous with stir bars were locked into place.

FIGS. 1A, 1B, and 1C show NMR spectra overlay of protected sulfonic acid synthetic route. FIG. 1A shows ¹H NMR of monomer 4, ethyl tricosa-1,22-diene-12-sulfonate in CDCl₃. FIG. 1B shows ¹H NMR of unsaturated ethyl protected polymer SO₃Et21U-33K in CDCl₃. FIG. 1C shows NMR of completely saturated ethyl protected polymer SO₃Et21-33K in CDCl₃.

Polymerizations in the bulk under high vacuum previously reached number-average molecular weights in the 20,000 g/mol range. By refluxing sulfonate monomers in DCM, molecular weights were improved significantly proving the ability of a refluxing ADMET solution polymerization to provide high molecular weights.

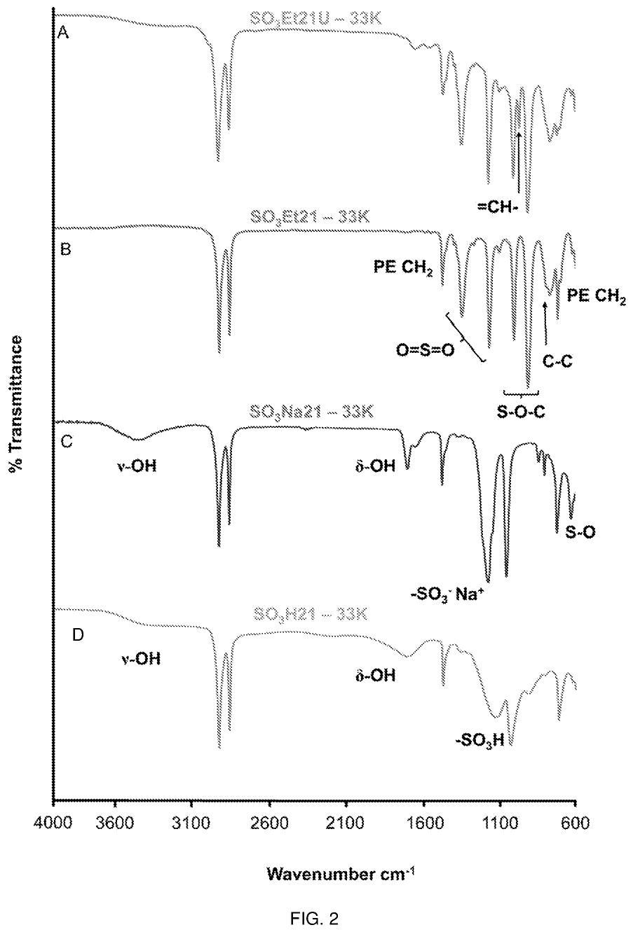

FIGS. 2A, 2B, 2C, and 2D show sulfonic polymer IR spectra overlay for each stage of the synthesis, representing each step of polymer transformation. FIG. 2A shows sulfonic polymer IR spectra for the SO₃Et21U-33K stage of the synthesis. FIG. 2B shows sulfonic polymer IR spectra for the SO₃Et21-33K stage of the synthesis. FIG. 2C shows sulfonic polymer IR spectra for the SO₃Na21-33K stage of the synthesis. FIG. 2D shows sulfonic polymer IR spectra for the SO₃H21-33K stage of the synthesis.

Catalytic hydrogenation was achieved with Wilkinson's catalyst at 500 psi of hydrogen gas and proceeded to completion for each polymer. Complete olefin conversion is apparent for SO₃Et21-33K in FIG. 1C, where no signals are present between 5 and 6 ppm. However, to further substantiate quantitative saturation, Fourier transform infrared spectroscopy (FT-IR) was performed. FIGS. 2A and 2B show IR spectra for SO₃Et21U-33K and SO₃Et21-33K, respectively. SO₃Et21U-33K exhibits an olefinic C—H wag at 967 cm⁻¹, which is clearly not present in SO₃Et21-33K.

All polymers exhibit polyethylene-like character evidenced by CH₂ scissoring (1464 cm⁻¹) and CH₂ rocking (721 cm⁻¹) modes in the IR spectra. SO₃Et21U-33K and SO₃Et21-33K each possess asymmetric O═S═O (1342 cm⁻¹) and symmetric O═S═O (1167 cm⁻¹) stretches. The ester protecting group in SO₃Et21U-33K and SO₃Et21-33K is definitively represented at 1005 cm⁻¹ and 912 cm⁻¹, with both signals indicative of S—O—C stretches.

Initially, deprotection was successful using a sodium hydroxide/DMSO solution for ester hydrolysis. The ester-protected polymer was suspended in DMSO, a poor solvent for the organic polymer. Upon the addition of sodium hydroxide pellets at 80° C., polymers were eventually reacted into solution as the esters were hydrolyzed. It was hypothesized that keeping the ester in solution ensured complete deprotection. This method was promising and worked well, but was abandoned due the difficulty of removing DMSO completely. Also, higher molecular weight species were not found to dissolve even after ester hydrolysis.

Alternatively, refluxing sodium methoxide and ethanol were found to deprotect all ester-protected polymers. Although the esters were not completely soluble, the reaction proceeded to completion. Sodium methoxide was chosen here, because a methoxide nucleophilic attack on the ether ester would produce methoxyethane, which boils at 7.4° C. and can be driven off easily at ˜80° C. to force the reaction to complete. The reaction is essentially a Williamson ether synthesis, in which the sulfonate moieties of the polymer are the leaving groups. After a 72-hour period, the sodium salt polymers were isolated. FIG. 2C (SO₃Na21-33K) contains no trace of sulfonate ester stretches at either 1005 cm⁻¹ or 912 cm⁻¹, strong evidence that this method results in complete deprotection. Further, SO₃Na21-33K exhibits a distinct S—O stretch (631 cm⁻¹) which is common of organic sulfonate compounds. After drying at elevated temperatures (˜100° C.) and high-vacuum, O—H stretching (3440 cm⁻¹) and O—H scissoring (1690 cm⁻¹) signals are present. These signals are not indicative of moisture, for which signals would be much broader. We believe these signals result from interactions between sulfonates and possible some sulfonic acids which may have already formed prior to acid treatment.

After sulfonate acidification with 12M hydrochloric acid in refluxing ethanol, the sulfonic acid polymer was isolated. SO₃Na21-33K and SO₃H21-33K show similar features via IR, but differ as follows (1) the S—O stretch associated with sulfonates (SO₃⁻) is not present in the IR of the acid: (2) the sulfonic stretch at 1123 cm⁻¹ lacks the intensity of the sodium sulfonate stretch; (3)O—H stretching and O—H scissoring differ slightly from acid to sodium salt. The different intensities associated with the acid stretching and bending are similar to that of previous carboxylic and phosphonic precision systems where 1:1 acid interactions where found between lamellae. Based on this preliminary IR data, we expect the same 1:1 acid behavior but this will require X-ray analysis as did the previous studies. The greater intensity of the sodium sulfonate stretch is caused by the stronger dipole-ion interactions occurring, which are not present in the sulfonic acid samples.

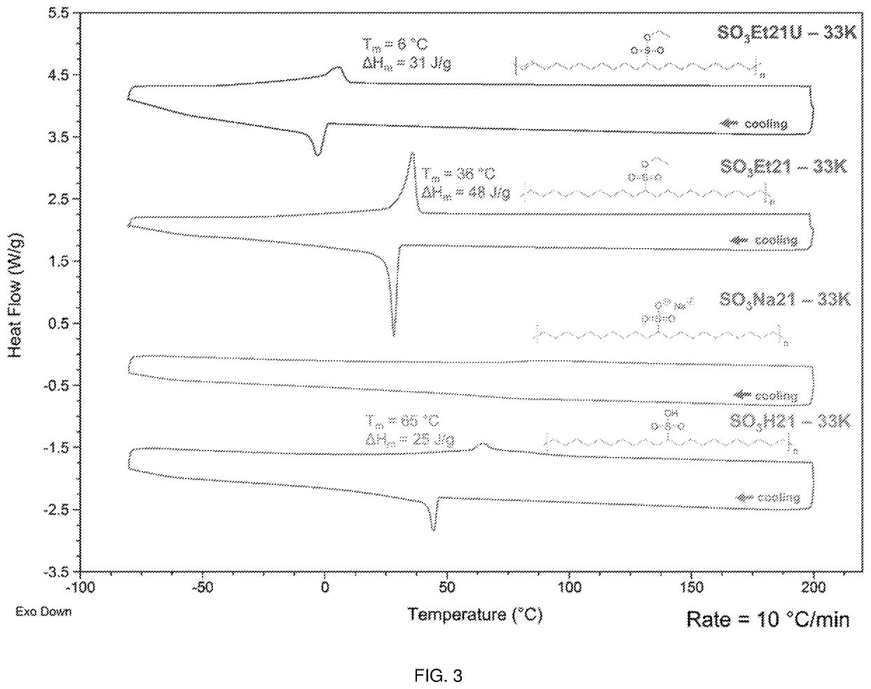

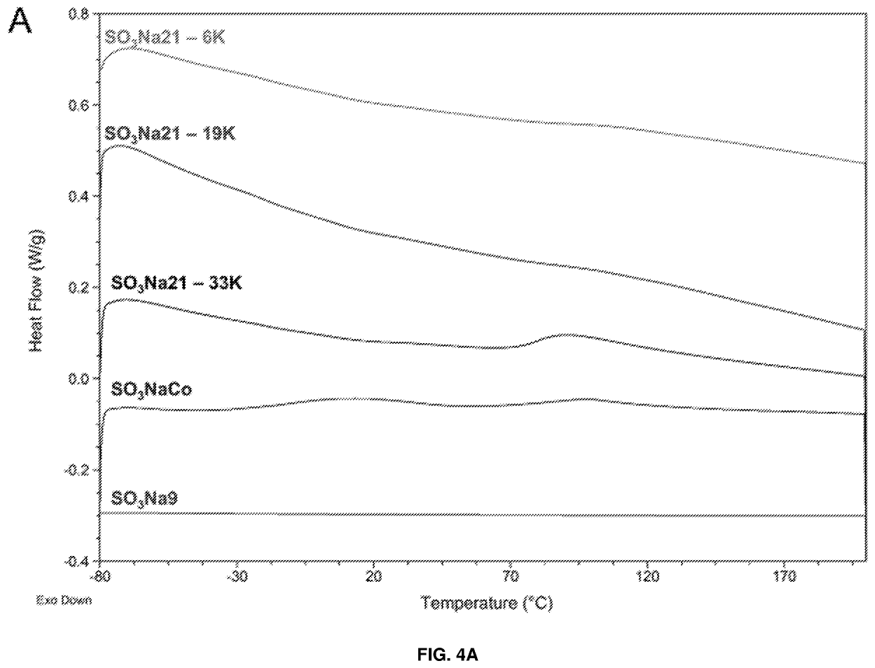

Semicrystallinity is observed in the DSC thermograms of SO₃Et21U-33K, SO₃Et21-33K, and SO₃H21-33K (FIG. 3). More specifically, FIG. 3 shows DSC thermogram overlay of SO₃Et21U-33K, SO₃Et21-33K, SO₃Na21-33K, and SO₃H21-33K representing each step of polymer transformation. Samples were heated/cooled at 10° C./min. In FIG. 3, the vertical scale is offset for clarity. Saturation of the internal olefins increases the Tₘ by 30° C. and the ΔHm by 17 J/g. Post-hydrogenation, such an increase is commonly observed and established in the literature for precision ADMET polymers. The lower melting points of unsaturated polymers are attributed to the existence of both cis and trans conformations, which disrupt crystallinity. SO₃Na21-33K isolated after the alkali deprotection step does not appear semicrystalline and exhibits no melt. Acidification of the sulfonates (R—SO₃⁻) to sulfonic acids (R—SO₃H) results in reversion to the previous crystalline nature but higher in melting point than the ethyl esters (65° C. for SO₃H21-33K compared to 36° C. for SO₃Et21-33K).

Similar salt-to-acid behavior was noted by Baughman. Precision acrylic acid copolymers with a carboxylic acid placed every 21^{st} melt at 45° C., while the corresponding zinc carboxylate copolymer did not melt before decomposition.

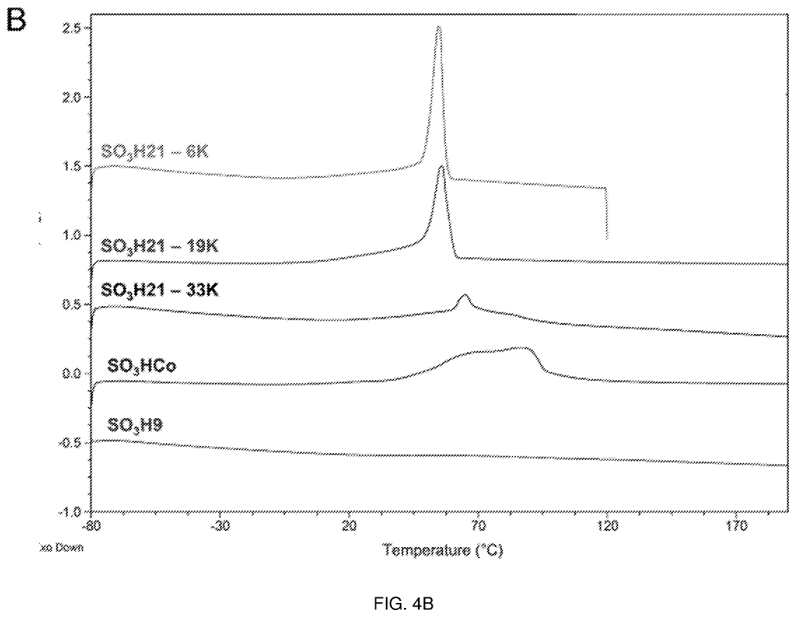

FIGS. 4A and 4B show DSC comparison of sodium sulfonate polymers vs. sulfonic acid polymers. FIG. 4A shows DSC thermogram overlay of sodium sulfonate polymers. FIG. 4B shows DSC thermogram overlay of sulfonic acid polymers. Samples were heated/cooled at 10° C./min. In FIGS. 4A and 4B, vertical scales are shifted for clarity.

As mentioned above, sodium sulfonate polymers (all carbon spacings, random and precise) do not exhibit semicrystallinity (FIG. 4A). However, upon acidification to the acid analog of each sample, crystallinity in longer run-length samples is regained. This coincides exactly with trends displayed by precision carboxylic and carboxylate samples prepared and characterized by Baughman and Seitz. The carboxylics have layered structures with hydrogen bonds between the carboxyl groups on adjacent layers; while the anions have ordered ionic cluster morphologies. These results suggest that the sulfonic polymers exhibit the same layered acid and ordered ionic cluster morphologies.

Each polymer containing a sulfonic acid on every 21^{st} carbon displays a fairly clear melt (FIG. 4B). As molecular weight is increased, melting temperatures are increased slightly (SO₃H21-6K<SO₃H21-33K<SO₃H21-33K). The random copolymer SO₃HCo containing an identical carbon to acid ratio melts over a broad range, indicating precision has a profound effect on the crystalline nature of the materials. SO₃H9 exhibits no distinct thermal transitions because the short run-lengths between acid groups prevent crystallization.

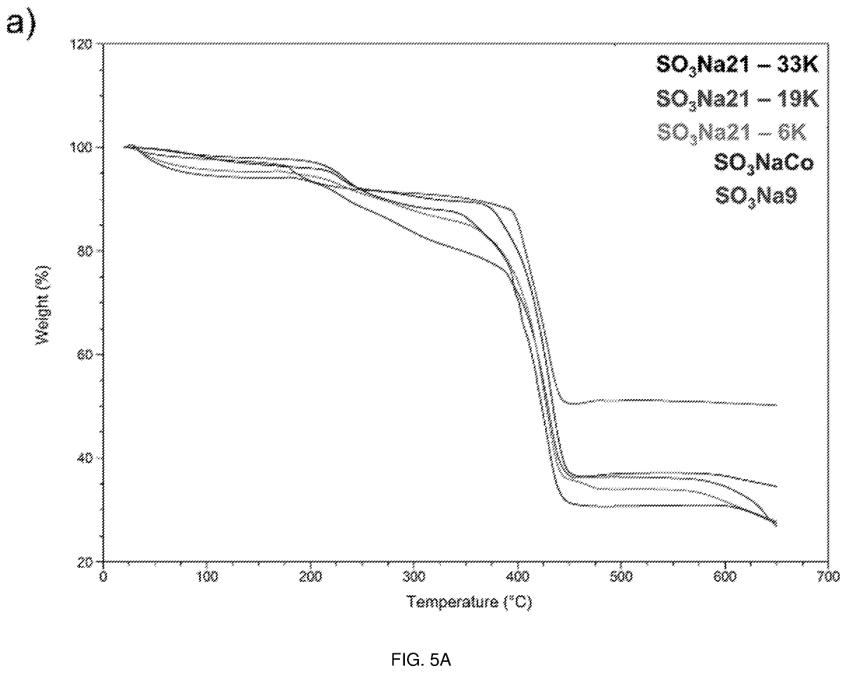

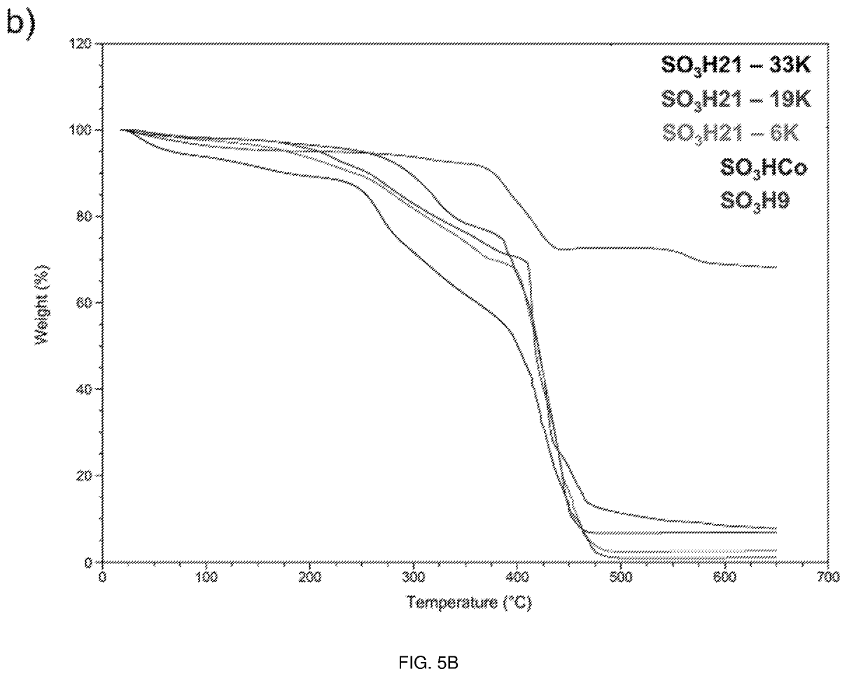

FIGS. 5A and 5B show TGA comparison of sodium sulfonate polymers vs. sulfonic acid polymers. FIG. 5A shows TGA thermogram overlay of sodium sulfonate polymers. FIG. 5B shows TGA thermogram overlay of sulfonic acid polymers. Samples were heated at 10° C./min.

Thermogravimetric analysis of all samples provides evidence of thermal stability to temperatures of around 200° C. Sodium sulfonate (FIG. 5A) and sulfonic acid (FIG. 5B) polymer samples appear to decompose within the same range as other sulfonated materials (˜280° C.). The initial weight losses correspond to desulfonation, known to occur first in sulfonated materials, followed by the degradation of the polyethylene backbone, typical in PE and other ADMET analogs.

Most of these precision sodium salts are clearly retaining more mass than their acid counterparts above 400° C., indicating the formation of ionic by-products from the sodium sulfonate. All samples exhibit good thermal stability and do not begin significant degradation until above 200° C., a temperature below which most potential applications will take place.

Opposed to hydrogenating samples post-ADMET polymerization as stated above, carbon-carbon double bonds within sulfone and sulfonate polymer backbones may be exploited to provide crosslinking. By reacting between 0.1% and 35% or between 0.1% and 30% of double bonds within polymer samples, a significant improvement in mechanical properties is observed. Combining such a mechanical improvement with the significant thermal properties afforded by the ADMET products will allow these materials to be used in a range of commodity and engineering applications in many forms including fibers and membranes.

Typical crosslinking reactions include, but are not limited to free-radical reactions, olefin metathesis with triene molecules, epoxidation followed by addition of various hardeners, thiol-ene and other “click” reactions. Essentially, any reaction to connect polymer chains through the usage of double bonds present in the sulfone and sulfonic polymers is employed.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### LIST OF REFERENCES

1. Piringer, O. G.; Baner, A. L.; Editors, Plastic Packaging; Interactions with Food and Pharmaceuticals. 2008; p 614 pp.
2. Schulz, M. D.; Wagener, K. B. Macromol. Chem. Phys. 2014, 215, (20), 1936-1945.
3. Opper, K. L.; Wagener, K. B. J. Polym. Sci., Part A Polym. Chem. 2011, 49, (4), 821-831.
4. Vasile, C.; Editor, Handbook of Polyolefins, Second Edition, Revised and Expanded. [In: Plast. Eng. (N. Y.), 2000; 59]. 2000; p 1014 pp.
5. Wagener, K. B.; Boncella, J. M.; Nel, J. G. Macromolecules 1991, 24, (10), 2649-57.
6. Harries-Rees, K.; Chauvin, Y.; Grubbs, R.; Schrock, R. Chem. World 2005, 2, (11), 42-44.
7. Lewin, M.; Editor, Handbook of Fiber Chemistry, Third Edition. [In: Int. Fiber Sci. Technol. Ser.; 2007, 16]. 2007; p 1044 pp.
8. Inci, B.; Wagener, K. B. J. Am. Chem. Soc. 2011, 133, (31), 11872-11875.
9. Calderon, N.; Ofstead, E. A.; Judy, W. A. J. Polym. Sci., Part A-1 Polym. Chem. 1967, 5, (9), 2209-17.
10. Schrock, R. R.; Hoveyda, A. H. Angew. Chem., Int. Ed. 2003, 42, (38), 4592-4633.
11. Anon, Handbook of Metathesis. by R. H. Grubbs. Wiley VCH: Weinheim. 2003. 1156 pp (3 volumes). $260. ISBN 3-527-30616-1. 2004; Vol. 8, p 294.
12. Herisson, J. L.; Chauvin, Y. Makromol. Chem. 1971, 141, 161-76.
13. Schulz, M. D.; Wagener, K. B. Abstracts of Papers, 243rd ACS National Meeting & Exposition, San Diego, Calif., United States, Mar. 25-29, 2012 2012, POLY-255.
14. Weychardt, H.; Plenio, H. Organometallics 2008, 27, (7), 1479-1485.
15. Schmidt, B. Eur. J. Org. Chem. 2004, (9), 1865-1880.
16. Boz, E.; Ghosal, A.; Alamo, R. G.; Wagener, K. B. Polym. Prepr. (Am. Chem. Soc., Div. Polym. Chem.) 2006, 47, (1), 219.
17. http://www.zimmernederland.nl/producten/heup/alternative-bearing-solutions/durasulr-highly-cross-linked-polyethylene/
18. Rojas, G.; Inci, B.; Wei, Y.; Wagener, K. B. J. Am. Chem. Soc. 2009, 131, (47), 17376-17386.
19. Smith, J. A.; Brzezinska, K. R.; Valenti, D. J.; Wagener, K. B. Macromolecules 2000, 33, (10), 3781-3794.
20. Sworen, J. C.; Smith, J. A.; Wagener, K. B.; Baugh, L. S.; Rucker, S. P. J. Am. Chem. Soc. 2003, 125, (8), 2228-2240.
21. Few, C. S.; Wagener, K. B.; Thompson, D. L. Macromol. Rapid Commun. 2014, 35, (2), 123-132.
22. Atallah, P.; Wagener, K. B.; Schulz, M. D. Macromolecules (Washington, D.C., U. S.) 2013, 46, (12), 4735-4741.
23. Schwendeman, J. E.; Wagener, K. B. Abstracts of Papers, 223rd ACS National Meeting, Orlando, Fla., United States, Apr. 7-11, 2002 2002, POLY-339.
24. Matsui, K.; Seno, S.; Nozue, Y.; Shinohara, Y.; Amemiya, Y.; Berda, E. B.; Rojas, G.; Wagener, K. B. Macromolecules (Washington, D.C., U. S.) 2013, 46, (11), 4438-4446.
25. Thompson, D. L.; Wagener, K. B.; Schulze, U.; Voit, B.; Jehnichen, D.; Malanin, M. Macromol. Rapid Commun. 2015, 36, (1), 60-64.
26. Valenti, D. J.; Wagener, K. B. Macromolecules 1998, 31, (9), 2764-2773.
27. Tuba, R.; Al-Hashimi, M.; Bazzi, H. S.; Grubbs, R. H. Macromolecules (Washington, D.C., U. S.) 2014, 47, (23), 8190-8195.
28. Spring, A. M.; Estrada, L. A.; Vasilyeva, S. V.; Rinzler, A. G.; Reynolds, J. R. Macromolecules (Washington, D.C., U. S.) 2014, 47, (8), 2556-2560.
29. Ortmann, P.; Heckler, I.; Mecking, S. Green Chem. 2014, 16, (4), 1816-1827.
30. Unverferth, M.; Meier, M. A. R. Polymer 2014, 55, (22), 5571-5575.
31. Ding, L.; Qiu, J.; Wei, J.; Zhu, Z. Polym. Chem. 2014, 5, (14), 4285-4292.
32. Ding, L.; Wei, J.; Qiu, J.; Wang, J.; Zhu, Z. RSC Adv. 2014, 4, (43), 22342-22349.
33. Parkhurst, R. R.; Balog, S.; Weder, C.; Simon, Y. C. RSC Adv. 2014, 4, (96), 53967-53974.
34. Kreye, O.; Trefzger, C.; Sehlinger, A.; Meier, M. A. R. Macromol. Chem. Phys. 2014, 215, (22), 2207-2220.
35. Baughman, T. W.; Chan, C. D.; Winey, K. I.; Wagener, K. B. Macromolecules (Washington, D.C., U. S.) 2007, 40, (18), 6564-6571.
36. Ortmann, P.; Trzaskowski, J.; Krumova, M.; Mecking, S. ACS Macro Lett. 2013, 2, (2), 125-127.
37. Seitz, M. E.; Chan, C. D.; Opper, K. L.; Baughman, T. W.; Wagener, K. B.; Winey, K. I. J. Am. Chem. Soc. 2010, 132, (23), 8165-8174.
38. Buitrago, C. F.; Opper, K. L.; Wagener, K. B.; Winey, K. I. ACS Macro Lett. 2012, 1, (1), 71-74.
39. Mader, J.; Xiao, L.; Schmidt, T. J.; Benicewicz, B. C. Adv. Polym. Sci. 2008, 216, (Fuel Cells II), 63-124.
40. Opper, K. L.; Fassbender, B.; Brunklaus, G.; Spiess, H. W.; Wagener, K. B. Macromolecules (Washington, D.C., U. S.) 2009, 42, (13), 4407-4409.
41. Opper, K. L.; Markova, D.; Klapper, M.; Mullen, K.; Wagener, K. B. Macromolecules (Washington, D.C., U. S.) 2010, 43, (8), 3690-3698.
42. Lebarbe, T.; More, A. S.; Sane, P. S.; Grau, E.; Alfos, C.; Cramail, H. Macromol. Rapid Commun. 2014, 35, (4), 479-483.
43. Shearouse, W. C.; Lillie, L. M.; Reineke, T. M.; Tolman, W. B. ACS Macro Lett. 2015, Ahead of Print.
44. Barbara, I.; Flourat, A. L.; Allais, F. Eur. Polym. J. 2015, 62, 236-243.
45. Llevot, A.; Grau, E.; Carlotti, S.; Grelier, S.; Cramail, H. Eur. Polym. J. 2014, Ahead of Print.
46. Tuzun, A.; Lligadas, G.; Ronda, J. C.; Galia, M.; Cadiz, V. Eur. Polym. J. 2015, Ahead of Print.
47. Leonard, J. K.; Hopkins, T. E.; Chaffin, K.; Wagener, K. B. Macromol. Chem. Phys. 2008, 209, (14), 1485-1494.
48. Leonard, J. K.; Wagener, K. B. Polym. Prepr. (Am. Chem. Soc., Div. Polym. Chem.) 2008, 49, (1), 874.
49. van Hensbergen, J. A.; Gaines, T. W.; Wagener, K. B.; Burford, R. P.; Lowe, A. B. Polym. Chem. 2014, 5, (21), 6225-6235.
50. Li, Z.-L.; Lv, A.; Du, F.-S.; Li, Z.-C. Macromolecules (Washington, D.C., U. S.) 2014, 47, (17), 5942-5951.
51. Wagener, K. B.; O'Donnell, P. M. Polym. Prepr. (Am. Chem. Soc., Div. Polym. Chem.) 2002, 43, (2), 48.
52. Simocko, C.; Young, T. C.; Wagener, K. B. Macromolecules (Washington, D.C., U. S.) 2015, 48, (16), 5470-5473.
53. Fisher, L. J. Sulfonic acid ionomers: Protection and deprotection schemes University of Florida, Gainesville, F L, 2010.
54. Opper, K. L. Polyethylene functionalized with highly polar groups. 2010.
55. Opper, K. L.; Wagener, K. B. Macromol. Rapid Commun. 2009, 30, (11), 915-919.
56. Boffa, L. S.; Novak, B. M. Chem. Rev. (Washington, D.C.) 2000, 100, (4), 1479-1493.
57. Kobayashi, S.; Müllen, K.; Aili, D.; Jensen, J.; Li, Q., Polymers for Fuel Cells. In Encyclopedia of Polymeric Nanomaterials, Springer Berlin Heidelberg: 2014; pp 1-13.
58. Piest, M., Boronic acid functionalized polymers and hydrogels for biomedical applications. 2011.
59. Cho, C.; Xiang, F.; Wallace, K. L.; Grunlan, J. C. Macromolecules (Washington, D.C., U. S.) 2015, 48, (16), 5723-5729.
60. Handbook of Smart Coatings for Materials Protection Elsevier: 2014.
61. Starostina, I., A.; Stoyanov, O., V.; Deberdeev, R. Y., Polymer Surfaces and Interfaces: Acid-Base Interactions and Adhesion in Polymer-Metal Systems. CRC Press: 2014.
62. Barbir, F., PEM Fuel Cells: Theory and Practice. Academic Press: 2013.
63. Mauritz, K. A.; Moore, R. B. Chem. Rev. (Washington, D.C., U. S.) 2004, 104, (10), 4535-4585.
64. Kucera, F.; Jancar, J. Polym. Eng. Sci. 1998, 38, (5), 783-792.
65. Fischer, D.; Eysel, H. H. J. Appl. Polym. Sci. 1994, 52, (4), 545-8.
66. Ireland, R. E.; Meissner, R. S. J. Org. Chem. 1991, 56, (14), 4566-8.
67. Pirrung, M. C., The Synthetic Organic Chemist's Companion. John Wiley & Sons: 2007.
68. Hrkach, J. S.; Matyjaszewski, K. Macromolecules 1990, 23, (18), 4042-6.
69. Nakanishi, K., Infrared Absorption Spectroscopy (Practical). Holden-Day, Inc.: San Francisco, 1962.
70. Mittal, V., High Performance Polymers and Engineering Plastics. John Wiley & Sons: 2011.
71. O'Gara, J. E.; Wagener, K. B.; Hahn, S. F. Makromol. Chem., Rapid Commun. 1993, 14, (10), 657-62.
72. Samms, S. R.; Wasmus, S.; Savinell, R. F. J. Electrochem. Soc. 1996, 143, (5), 1498-1504.
73. Aitken, B. S.; Buitrago, C. F.; Heffley, J. D.; Lee, M.; Gibson, H. W.; Winey, K. I.; Wagener, K. B. Macromolecules (Washington, D.C., U. S.) 2012, 45, (2), 681-687.
74. van Woerden, H. F. Chem. Rev. 1960, 63, (6), 557-71.
75. McCormack, W. B.; Lawes, B. C. Kirk-Othmer Encycl. Chem. Technol., 3rd Ed. 1983, 22, 233-54.
76. Guenther, A.; Koenig, T.; Habicher, W. D.; Schwetlick, K. Polym. Degrad. Stab. 1997, 55, (2), 209-216.
77. Mao, L.; Li, B.; Cui, X.; Zhao, Y.; Xu, X.; Shi, X.; Li, S.; Li, F. Electrochim. Acta 2012, 79, 197-201.
78. Buitrago, C. F.; Alam, T. M.; Opper, K. L.; Aitken, B. S.; Wagener, K. B.; Winey, K. I. Macromolecules (Washington, D.C., U. S.) 2013, 46, (22), 8995-9002.
79. Heiney, P. A., Datasqueeze: A Software Tool for Powder and Small-Angle X-Ray Diffraction Analysis. Comun. Powder Diff. Newsl., 2005; Vol. 32, pp 9-11.
80. Weixler, R.; Hehn, J. P.; Bach, T. J. Org. Chem. 2011, 76, (15), 5924-5935.
81. Kiasat, A. R.; Kazemi, F.; Khosravian, F. Phosphorus, Sulfur Silicon Relat. Elem. 2004, 179, (2), 427-431.
82. Seto, T.; Hara, T.; Tanaka, K. Jap. J. Appl. Phys. 1968, 7, (1), 31-42.
83. Aitken, B. S.; Wieruszewski, P. M.; Graham, K. R.; Reynolds, J. R.; Wagener, K. B. Macromolecules (Washington, D.C., U. S.) 2012, 45, (2), 705-712.
84. Berda, E. B.; Wagener, K. B. Synth. Polym. 2012, 2, 587-599.
85. Few, C. S.; Williams, K. R.; Wagener, K. B. Tetrahedron Lett. 2014, 55, (32), 4452-4454.
86. Boz, E.; Wagener, K. B.; Ghosal, A.; Fu, R.; Alamo, R. G. Macromolecules 2006, 39, (13), 4437-4447.
87. Boz, E.; Nemeth, A. J.; Ghiviriga, I.; Jeon, K.; Alamo, R. G.; Wagener, K. B. Macromolecules (Washington, D.C., U. S.) 2007, 40, (18), 6545-6551.
88. Thompson, D.; Yamakado, R.; Wagener, K. B. Macromol. Chem. Phys. 2014, 215, (12), 1212-1217.
89. Bachler, P. R.; Wagener, K. B. Monatsh. Chem. 2015, 146, (7), 1053-1061.
90. Gaines, T. W.; Nakano, T.; Chujo, Y.; Trigg, E. B.; Winey, K. I.; Wagener, K. B. ACS Macro Lett. 2015, 4, (6), 624-627.
91. El-Hibri, M. J.; Weinberg, S. A. Encycl. Polym. Sci. Technol. (4th Ed.) 2014, 11, 179-204.
92. Faye, A.; Leduc, M.; Brisson, J. Polym. Chem. 2014, 5, (7), 2548-2560.
93. Parker, D.; Bussink, J.; van de Grampel, H. T.; Wheatley, G. W.; Dorf, E.-U.; Ostlinning, E.; Reinking, K.; Schubert, F.; Jünger, O.; Wagener, R., Polymers, High-Temperature. Ullmann's Encyclopedia of Industrial Chemistry. Wiley-VCH: Weinheim: 2012.
94. Ellis, B.; Smith, R., Polymers—A Property Database. 2nd ed.; Taylor & Francis:

2009; pp 325-326.
95. Chen, A.; Wu, D.; Johnson, C. S., Jr. J. Am. Chem. Soc. 1995, 117, (30), 7965-70.
96. Rojas, G.; Inci, B.; Wei, Y.; Wagener Kenneth, B. J Am Chem Soc 2009, 131, (47), 17376-86.
97. Furniss, B. S.; Hannaford, A. J.; Smith, P. W. G.; Tatchell, A. R., Vogel's Textbook of Practical Organic Chemistry. 5th ed.; Harlow: Longman: 1989.
98. Bahrami, K.; Khodaei, M. M.; Sheikh Arabi, M. J. Org. Chem. 2010, 75, (18), 6208-6213.
99. Schulz, M. D.; Wagener, K. B. ACS Macro Lett. 2012, 1, (4), 449-451. 100. Buitrago, C. F.; Jenkins, J. E.; Opper, K. L.; Aitken, B. S.; Wagener, K. B.; Alam, T. M.; Winey, K. I. Macromolecules (Washington, D.C., U. S.) 2013, 46, (22), 9003-9012.
101. Alger; M., M. S., Polymer Science Dictionary. Chapman & Hall: 1997; p 344-346.
102. Kappe, C. O.; Dallinger, D.; Murphree, S. S.; Editors, Practical Microwave Synthesis for Organic Chemists: Strategies, Instruments, and Protocols. 2009; p 299 pp.
103. Kappe, C. O. Angew. Chem., Int. Ed. 2004, 43, (46), 6250-6284.
104. Nakamura, T.; Nagahata, R.; Takeuchi, K. Mini-Rev. Org. Chem. 2011, 8, (3), 306-314.
105. Yamada, S.; Takasu, A.; Takayama, S.; Kawamura, K. Polym. Chem. 2014, 5, (18), 5283-5288.
106. Giachi, G.; Frediani, M.; Rosi, L.; Frediani, P. Microwave Heat. 2011, 181-206.
107. Brooks, W. L. A.; Sumerlin, B. S. Isr. J. Chem. 2012, 52, (3-4), 256-263.
108. Singla, P.; Mehta, R.; Berek, D.; Upadhyay, S. N. J. Macromol. Sci., Part A Pure Appl. Chem. 2014, 51, (4), 350-361.
109. Zhu, X.; Chen, J.; Zhou, N.; Cheng, Z.; Lu, J. Eur. Polym. J. 2003, 39, (6), 1187-1193.
110. Costa, C.; Santos, A. F.; Fortuny, M.; Araujo, P. H. H.; Sayer, C. Mater. Sci. Eng., C 2009, 29, (2), 415-419.

What is claimed is:

1. A poly(sulfonic acid) comprising a multiplicity of sulfonic acid units separated by alkylene units in a polymer chain or a copolymer chain, the poly(sulfonic acid) having a degree of crosslinking in a range of from about 0.1 to about 30 percent.

2. The poly(sulfonic acid) according to claim 1, wherein on average at least two alkylene units of each polymer chain or copolymer chain comprise a crosslinking unit between at least two polymer chains or copolymer chains.

3. The poly(sulfonic acid) according to claim 2, wherein the crosslinking unit comprises the reaction product of an ethenylene unit with a diacrylate or a dithiol.

4. The poly(sulfonic acid) according to claim 2, wherein the crosslinking unit comprises the reaction product of an epoxy unit formed from an ethenylene unit and a diol or a diamine.

5. The poly(sulfonic acid) according to claim 1, wherein the alkylene units are of the same mass and/or structure.

6. The poly(sulfonic acid) according to claim 1, wherein the alkylene units are of at least three different masses and/or structures.

7. The poly(sulfonic acid) according to claim 1, wherein the alkylene unit is a C₄ to C₃₆ unit.

8. The poly(sulfonic acid) according to claim 1, wherein the alkylene unit consists of a multiplicity of methylene units.

9. The poly(sulfonic acid) according to claim 1, where at least one of the alkylene units further comprises an ethenylene unit separated from the sulfone units by at least one methylene unit.

10. The poly(sulfonic acid) according to claim 6, wherein each of the alkylene units consists of the ethenylene unit separated from the sulfone units by at least one methylene unit.

11. A membrane comprising a poly(sulfonic acid) according to claim 1.

12. A fuel cell comprising a poly(sulfonic acid) according to claim 1.

13. A gas barrier comprising a poly(sulfonic acid) according to claim 1.

14. A method of preparing a poly(sulfonic acid) having improved mechanical integrity, the method comprising:
synthesizing a poly(sulfonic acid) by acyclic diene metathesis (ADMET) polymerization; and
reacting a plurality of double bonds afforded by the ADMET polymerization with a crosslinker to achieve a degree of crosslinking in a range of from about 0.1 to about 30 percent.

15. A method comprising:
polymerizing an ethyl-protected sulfonate ester diene monomer via ADMET polymerization,
wherein the ethyl-protected sulfonate ester diene monomer, has a structure, 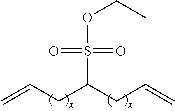
in which x is from 1 to 25,
to produce a polymer, having a structure, 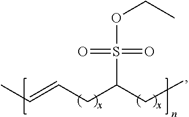
in which x is from 1 to 25, and n is from 1 to 5000.

16. The method, according to claim 15, further comprising hydrogenating the polymer to produce a saturated polymer, having a structure, 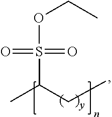
in which x is from 1 to 25, and n is from 1 to 5000.

17. The method according to claim 16, further comprising
deprotecting the saturated polymer to produce a deprotected sulfonate polymer; and
acidifying the deprotected sulfonate polymer to produce a poly(sulfonic acid), having a structure, 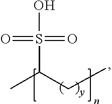
in which y is from 2 to 100, and n is from 1 to 5000.

18. The method according to claim 17, wherein deprotecting the saturated polymer to produce the deprotected sulfonate polymer comprises contacting the saturated polymer with a polar solvent and one selected from the group consisting of sodium methoxide, potassium hydroxide, sodium hydroxide, and combinations thereof.

19. The method, according to claim 15, further comprising
deprotecting the polymer to produce a deprotected sulfonate polymer; and
acidifying the deprotected sulfonate polymer to produce a poly(sulfonic acid), having a structure, 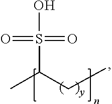
in which y is from 2 to 100, and n is from 1 to 5000.

20. The method according to claim 19, wherein deprotecting the polymer to produce the deprotected sulfonate polymer comprises contacting the polymer with a polar solvent and one selected from the group consisting of sodium methoxide, potassium hydroxide, sodium hydroxide, and combinations thereof.

21. The method according to claim 19, further comprising crosslinking the poly(sulfonic acid).

22. The method according to claim 15, further comprising producing the ethyl-protected sulfonate ester diene monomer by reacting an alkenol, having a structure, 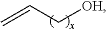
in which x is from 1 to 25,
with a trifluoromethanesulfonic anhydride, having a structure, 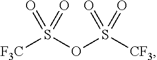 [SMILES: O=S(=O)(OS(=O)(=O)C(F)(F)F)C(F)(F)F]
to produce a triflate functionalized alkene species, having a structure, 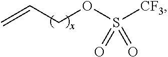
in which x is from 1 to 25;
reacting the triflate functionalized alkene species with a deprotonated ethyl methane sulfonate to produce the ethyl-protected sulfonate ester diene monomer.
